SEQUENCE LISTING (67 sequences)

SEQ ID NO: 1 - protein, 5 aa, Artificial Sequence
  Consensus amino acid sequence
XXXPX

SEQ ID NO: 2 - protein, 5 aa, Saccharum officinarum
IRLPS

SEQ ID NO: 3 - protein, 5 aa, Saccharum officinarum
IKLPS

SEQ ID NO: 4 - protein, 5 aa, Saccharum officinarum
LRLPS

SEQ ID NO: 5 - protein, 5 aa, Saccharum officinarum
LKLPS

SEQ ID NO: 6 - protein, 88 aa, Saccharum officinarum
MVTARLRLALLLLSVFLCSAWARPRLEPTIRLPSERAAAAAGDETDDAVGTRWAVLVAGSSGYYNYRHQADICHAYQIMKKGGLKDEN

SEQ ID NO: 7 - protein, 23 aa, Saccharum officinarum
LCSAWARPRLEPTIRLPSERAAA

SEQ ID NO: 8 - protein, 25 aa, Saccharum officinarum
RPRLEPTIRLPSERAAAAAGDETDD

SEQ ID NO: 9 - protein, 22 aa, Saccharum officinarum
MVTARLRLALLLLSVFLCSAWA

SEQ ID NO: 10 - protein, 488 aa, Saccharum officinarum
MVTARLRLALLLLSVFLCSAWARPRLEPTIRLPSERAAAAAGDETDDAVGTRWAVLVAGSSGYYNYRHQADICHAYQIMKKGGLKDENIIVFMYDDIAHSAENPRPGVVINHPQGGDVYAGVPKDYTGRQVSVNNFFAVLLGNKTALTGGSGKVVDSGPNDHIFVFYSDHGGPGVLGMPTYPYLYGDDLVDVLKKKHAAGSYKSLVFYLEACESGSIFEGLLPDDINVYATTASNAEESSWGTYCPGEFPSPPPEYDTCLGDLYSVSWMEDSDFHNLRTESLKQQYKLVKDRTAAQDTFSYGSHVMQYGSLELNVQKLFSYIGTNPANDGNTFVEDNSLPSFSKAVNQRDADLVYFWQKYRKLADGSSKKNEARKELLEVMSHRSHVDNSVELIGSLLFGSEDGPRVLKAVRAAGEPLVDDWSCLKSMVRTFEAQCGSLAQYGMKHMRTFANICNAGILPEAVSKVAAQACTSIPSNPWSSIDKGFSA

SEQ ID NO: 11 - protein, 485 aa, Zea mays
MVADRLRLALLLSACLCSAWARPRLEPTIRLPSERAAADETDDDAVGTRWAVLIAGSNGYYNYRHQADICHAYQIMKKGGLKDENIVVFMYDDIAHSPENPRPGVIINHPQGGDVYAGVPKDYTGRDVNVDNFFAVLLGNKTALRGGSGKVVDSGPNDHIFVFYSDHGGPGVLGMPTYPYLYGDDLVDVLKKKHAAGTYKSLVFYLEACESGSIFEGLLPNDINVYATTASNAEESSWGTYCPGEFPSPPPEYDTCLGDLYSVAWMEDSDFHNLRTESLKQQYKLVKDRTAAQDTFSYGSHVMQYGSLELNVQKLFSYIGTNPANDGNTFIEDNSLPSLSKAVNQRDADLVYFWQKYRKLADSSPEKNEARRELLEVMAHRSHVDSSVELIGSLLFGSEDGPRVLKAVRAAGEPLVDDWSCLKSTVRTFEAQCGSLAQYGMKHMRSFANICNAGILPEAVSKVAAQACTSIPSNPWSSIHKGFSA

SEQ ID NO: 12 - protein, 501 aa, Oryza sativa
MAARARLRLVLPPLAALLLFAHLAAVAVARPRWEEEGSNLRLPSERAVAAGAAADDAAEAAEGTRWAVLIAGSNGYYNYRHQADVCHAYQIMKRGGLKDENIIVFMYDDIAHNPENPRPGVIINHPQGGDVYAGVPKDYTGKEVNVKNLFAVLLGNKTAVKGGSGKVLDSGPNDHIFIFYSDHGGPGVLGMPTYPYLYGDDLVDVLKKKHAAGTYKSLVFYLEACESGSIFEGLLPNGINVYATTASNADESSWGTYCPGEYPSPPPEYDTCLGDLYSVAWMEDSDVHNLRTESLKQQYNLVKDRTAVQDTFSYGSHVMQYGSLELNVKHLFSYIGTNPANDDNTFVEDNSLPSFSRAVNQRDADLVYFWQKYRKLPESSPEKNEARKQLLEMMAHRSHVDNSVELIGNLLFGSEEGPRVLKAVRATGEPLVDDWSCLKSMVRTFEAQCGSLAQYGMKHMRSFANICNAGISAEAMAKVAAQACTSIPSNPWSSTHRGFSA

SEQ ID NO: 13 - protein, 491 aa, Arabidopsis thaliana
MTRVSVGVVLFVLLVSLVAVSAARSGPDDVIKLPSQASRFFRPAENDDDSNSGTRWAVLVAGSSGYWNYRHQADICHAYQLLRKGGLKEENIVVFMYDDIANNYENPRPGTIINSPHGKDVYQGVPKDYTGDDVNVDNLFAVILGDKTAVKGGSGKVVDSGPNDHIFIFYSDHGGPGVLGMPTSPYLYANDLNDVLKKKHALGTYKSLVFYLEACESGSIFEGLLPEGLNIYATTASNAEESSWGTYCPGEEPSPPPEYETCLGDLYSVAWMEDSGMHNLQTETLKQQYNLVKERTSVQHTYYSGSHVMEYGSLELNAHHVFMYMGSNPANDNATFADANSLKPPSRVTNQRDADLVHFWEKYRKAPEGSARKTEAQKQVLEAMSHRLHIDNSVILVGKILFGISRGPEVLNKVRSAGQPLVDDWNCLKNQVRAFERHCGSLSQYGIKHMRSFANICNAGIQMEQMEEAASQACTTLPTGPWSSLNRGFSA

SEQ ID NO: 14 - protein, 489 aa, Nicotiana tabacum
MIRYVAGTLFLIGLALNVAVSESRNVLKLPSEVSRFFGADESNAGDHDDDSVGTRWAILLAGSNGYWNYRHQADICHAYQLLKKGGLKDENIVVFMYDDIANNEENPRRGVIINSPHGEDVYKGVPKDYTGDDVTVDNFFAVILGNKTALSGGSGKVVNSGPNDHIFIFYSDHGGPGVLGMPTDPYLYANDLIDVLKKKHASGTYKSLVFYLEACESGSIFEGLLPEGLNIYATTASNAEESSWGTYCPGEYPSPPIEYMTCLGDLYSISWMEDSELHNLRTESLHQQYELVKRRTAPVGYSYGSHVMQYGDVGISKDNLDLYMGTNPANDNFTFMDDNSLRVSKAVNQRDADLLHFWHKFRTAPEGSVRKIEAQKQLNEAISHRVHLDNSVALVGKLLFGIEKGPEVLSGVRPAGQPLVDDWDCLKSFVRTFETHCGSLSQYGMKHMRSIANICNAGIKKEQMVEASAQACPSVPSNTWSSLHRGFSA

SEQ ID NO: 15 - protein, 495 aa, Citrus sinensis
MTRLASGVLITLLVALAGIADGSRDIAGDILKLPSEAYRFFHNGGGGAKVNDDDDSVGTRWAVLLAGSNGFWNYRHQADICHAYQLLRKGGLKDENIIVFMYDDIAFNEENPRPGVIINHPHGDDVYKGVPKDYTGEDVTVEKFFAVVLGNKTALTGGSGKVVDSGPNDHIFIFYSDHGGPGVLGMPTSRYIYADELIDVLKKKHASGNYKSLVFYLEACESGSIFEGLLLEGLNIYATTASNAEESSWGTYCPGEIPGPPPEYSTCLGDLYSIAWMEDSDIHNLRTETLKQQYHLVKERTATGNPVYGSHVMQYGDLHLSKDALYLYMGTNPANDNYTFVDENSLRPASKAVNQRDADLLHFWDKYRKAPEGTPRKAEAQKQFFEAMSHRMHVDHSIKLIGKLLFGIEKGPEILNTVRPAGQPLVDDWGCLKSLVRTFESHCGALSQYGMKHMRSLANICNTGIGKEKMAEASAQACENIPSGPWSSLDKGFSA

SEQ ID NO: 16 - protein, 433 aa, Xenopus laevis
MFLHLAALVSFVLGASSVPFSNPEDTGKHWVVLVAGSNGWYNYRHQADVCHAYQIVKKNGIPDEQIVVMMYDDIANNDENPTKGVIINRPNGTDVYAGVLKDYIGDDVNPKNFLAVLSGDSEAVKGKGSGKVIRSGPNDHVFVYFTDHGAPGLLAFPSDDLHVMELNKTIQHMYENKKYKKMVFYIEACESGSMMNHLPNNINVYATTAANPQESSYACYYDDKRDTYLGDLYSVSWMEDSDMEDLAKETLHKQFVLVKQHTNTSHVMQYGNRTISQMKVNQFQGNVKITSTPVYLEPVKHMDLTPSPDVPLAILKRKLMATNDILQARAIVREIKAHQEAKQLIKESMRKIVNMVTESDELTEEILTDQVIINDTQCYRDAAEHFKRQCFNWHNPLYEYALRNLYALVNLCESGYPIERVHKAMEKVCNSWN

SEQ ID NO: 17 - protein, 435 aa, Rattus norvegicus
MIWKVAVLLSLVLGAGAVHIGVDDPEDGGKHWVVIVAGSNGWYNYRHQADACHAYQIIHRNGIPDEQIIVMMYDDIANNEENPTPGVVINRPNGTDVYKGVPKDYTGEDVTPENFLAVLRGDEEAVKGKGSGKVLKSGPRDHVFVYFTDHGATGILVFPNEDLHVKDLNKTIRYMYEHKMYQKMVFYIEACESGSMMNHLPDDIDVYATTAANPNESSYACYYDEERSTYLGDWYSVNWMEDSDVEDLTKETLHKQYHLVKSHTNTSHVMQYGNKSISTMKVMQFQGMKHRASSPISLPPVTHLDLTPSPDVPLTILKRKLLRTNNMKESQVLVGQIQHLLDARHIIEKSVQKIVSLLAGFGETAQKHLSERAMLTAHDCHQEAVTHFRTHCFNWHSVTYEHALRYLYVLANLCEKPYPIDRIKMAMDKVCLSHY

SEQ ID NO: 18 - protein, 433 aa, Bos taurus
MIWEFTVLLSLVLGTGAVPLEDPEDGGKHWVVIVAGSNGWYNYRHQADACHAYQIVHRNGIPDEQIIVMMYDDIANSEDNPTPGIVINRPNGSDVYQGVLKDYTGEDVTPKNFLAVLRGDAEAVKGVGSGKVLKSGPRDHVFVYFTDHGATGILVFPNEDLHVKDLNETIRYMYEHKMYQKMVFYIEACESGSMMNHLPPDINVYATTAANPRESSYACYYDEQRSTFLGDWYSVNWMEDSDVEDLTKETLHKQYQLVKSHTNTSHVMQYGNKSISAMKLMQFQGLKHQASSPISLPAVSRLDLTPSPEVPLSIMKRKLMSTNDLQESRRLVQKIDRHLEARNIIEKSVRKIVTLVSGSAAEVDRLLSQRAPLTEHACYQTAVSHFRSHCFNWHNPTYEYALRHLYVLVNLCENPYPIDRIKLSMNKVCHGYY

SEQ ID NO: 19 - protein, 433 aa, Homo sapiens
MVWKVAVFLSVALGIGAVPIDDPEDGGKHWVVIVAGSNGWYNYRHQADACHAYQIIHRNGIPDEQIVVMMYDDIAYSEDNPTPGIVINRPNGTDVYQGVPKDYTGEDVTPQNFLAVLRGDAEAVKGIGSGKVLKSGPQDHVFIYFTDHGSTGILVFPNEDLHVKDLNETIHYMYKHKMYRKMVFYIEACESGSMMNHLPDNINVYATTAANPRESSYACYYDEKRSTYLGDWYSVNWMEDSDVEDLTKETLHKQYHLVKSHTNTSHVMQYGNKTISTMKVMQFQGMKRKASSPVPLPPVTHLDLTPSPDVPLTIMKRKLMNTNDLEESRQLTEEIQRHLDARHLIEKSVRKIVSLLAASEAEVEQLLSERAPLTGHSCYPEALLHFRTHCFNWHSPTYEYALRHLYVLVNLCEKPYPLHRIKLSMDHVCLGHY

SEQ ID NO: 20 - protein, 46 aa, Saccharum officinarum
MRPAGQLLLPLLLLAVAASAAGSWEDGHGSILRLPSSSSPRRFPRS

SEQ ID NO: 21 - protein, 51 aa, Saccharum officinarum
MGTIPVVIPAMLVVALLVVGATATATAAAARPSHTDTDAIRLPSNAGAGGR

SEQ ID NO: 22 - protein, 47 aa, Saccharum officinarum
MRMGHRTCGTIFILLYVISTSTLLASSNEHGLIRIPLKKRSIMDSIY

SEQ ID NO: 23 - protein, 27 aa, Saccharum officinarum
WARPRLEPTIRLPSERAAAAAGDETDD

SEQ ID NO: 24 - protein, 5 aa, Artificial sequence
  Less variable consensus sequence
XXLPS

SEQ ID NO: 25 - protein, 117 aa, Saccharum officinarum
EARKELLEVMSHRSHVDNSVELIGSLLFGSEDGPRVLKAVRAAGEPLVDDWSCLKSMVRTFEAQCGSLAQYGMKHMRTFANICNAGILPEAVSKVAAQACTSIPSNPWSSIDKGFSA

SEQ ID NO: 26 - DNA, 20 bp, Artificial sequence
  Synthetic primer sequence
cgtctcgccttctttcgtcc

SEQ ID NO: 27 - DNA, 25 bp, Artificial sequence
  Synthetic PCR primer
tgtaatgtaatggagttcggtgtgg

SEQ ID NO: 28 - DNA, 30 bp, Artificial sequence
  Synthetic primer sequence
gcgggatccgcgtctcgccttctttcgtcc

SEQ ID NO: 29 - DNA, 31 bp, Artificial sequence
  Synthetic primer sequence
gtgctaccatggcctcgtccttgagtcctcc

SEQ ID NO: 30 - DNA, 1467 bp, Saccharum officinarum
atggtgaccgctcgcctccgcctcgcgctgctactactctccgtgttcctctgctccgcgtgggcgcgcccacgcctcgagccgaccatccgcctgccgtccgagcgcgccgcggcggcggccggcgacgaaacggacgacgccgtcgggacacggtgggccgtgctcgtcgccggctccagcggctactacaactaccgccaccaggcagacatctgccatgcgtaccagattatgaagaagggaggactcaaggacgagaacataattgtcttcatgtacgatgacatcgcgcatagcgcagagaatccgaggcccggtgtcgtcatcaaccatccccagggtggcgatgtctatgctggggttccaaaggattacactgggcgacaggtcagtgtcaacaatttcttcgctgttctgcttggcaacaaaactgctctgacaggtgggagcggcaaggttgtggacagtggccccaatgatcatatctttgttttctacagtgaccatggaggtcctggtgtccttggaatgcctacgtatccatatctctacggtgatgacctcgtagatgtcctgaagaagaagcatgctgctgggtcctacaaaagcctggtcttttaccttgaagcatgcgaatctgggagcatctttgagggcctcctgccagatgacatcaatgtgtatgcgaccaccgcgtcaaatgcagaggagagcagctgggggacgtactgccctggcgagttcccaagccctccaccggagtatgacacttgcttgggagacctgtatagtgtttcttggatggaagacagtgatttccacaatctgcgaactgaatctctcaagcagcagtacaagttggtcaaggataggacagcggctcaggatacattcagctatggttcccatgtgatgcaatatggttcattggagttgaatgttcagaaattgttttcgtacattggcacaaaccctgctaacgatggcaacacatttgtagaagataactcattgccatcattttcaaaagctgttaatcagcgtgatgctgatcttgtctacttctggcagaagtaccggaaattggctgatggctcatctaagaaaaatgaagctcggaaggaattgcttgaagtgatgtcccaccggtctcatgttgacaacagtgttgaactcattggaagccttctctttggctctgaggacggtccaagggttctgaaagccgtccgtgcagctggtgaacctctggttgatgattggagttgcctcaagtccatggttcgtacttttgaggcgcaatgtgggtcgttggcgcagtatgggatgaagcacatgagaaccttcgcaaacatctgcaacgctggcatccttcctgaagcagtgtcaaaggttgccgctcaggcttgcaccagcattccttccaacccctggagctctatcgacaagggttttagcgcctaa

SEQ ID NO: 31 - DNA, 868 bp, Saccharum officinarum
attaaccgattcacttccaccactccaccgccaccacctgtcgccggaccagaccagccatgcgtcccgccggccagctactcctcccgctcctcctcctcgccgtctccgtcgcagccgccgccggcggcggctcgtgggaggacggccatgggagcatcctccgcctcccctcctcctcctccccgcggcggttcccccgctccgccgccgtcgacctgatccgcgcgctgaacctccaccccgccgacgcctccccgcccctctccaccgccggcgtcgagggcgccctcgcccccgcggggaccctcgtcgagaggcccatccgcctcgcgtccttcgcggacgcaggcgacgccggcacgtcggtggaggacctcggccaccacgcggggtactaccgcctccccaacacccacgacgccaggatgttctacttcttcttcgagtcgaggggccaggaggacgaccccgtggtgatctggctcacgggcgggcccggctgcagcagcgagctcgcgctcttctacgagaacggccccttcaacatagcagacaacttgtcgctcgtctggaatgatttccgttgggacaaggcatcaaatcttatctatgttgaccagcccactggaactgggtttagctacagctcggactcgcgtgacactcgccacaacgaagctactattagcaatgatctatatggactttctgcaggcctttttaactgagcaccaaaggatggctaaaatgatttcttataacccgggaatatatgcggggcattacattcctgcctttgcaaggcgtgttgcacaagggaaacagaacaatgagggcttcacctttaacttgaagggttttgcga

SEQ ID NO: 32 - protein, 223 aa, Saccharum officinarum
MRPAGQLLLPLLLLAVSVAAAAGGGSWEDGHGSILRLPSSSSPRRFPRSAAVDLIRALNLHPADASPPLSTAGVEGALAPAGTLVERPIRLASFADAGDAGTSVEDLGHHAGYYRLPNTHDARMFYFFFESRGQEDDPVVIWLTGGPGCSSELALFYENGPFNIADNLSLVWNDFRWDKASNLIYVDQPTGTGFSYSSDSRDTRHNEATISNDLYGLSAGLFN

SEQ ID NO: 33 - DNA, 1114 bp, Saccharum officinarum
cacgagggcacgccgacaccgtgtaccggcctagctagcactcaactcaacttcatcggacttgcagaaagacgaagcagagcagcggcatgggcaccatccccgtcgtcatccccgcgatgctcgtcgtcgcccttcttgtcgtcggcgccaccgccaccgccaccgccaccgccgccagacccagccacaccgacaccgaagcgattcgtctccccagcagcactgctgctggtcaaccgtgggagtgctgcgactgggttacccaggacccgacgttcaggccgccgaagtggcgctgcaacgacgtggtggacaagtgctccgccgactgccagcagtgcgaggcgtcggcggccggcgacggcttcgtctgccgtgactggatcttcagcctgttcgagcccccggtctgcacaccgaggccatgggactgctgcgacctcgccgcctgcaccagggactacatcccctactgccggtgcgccgacgaggtggagtcgtgcccgagcaactgcaaagagtgcgagctcgtggagtcggaggagtcggaccctcctcgctaccgttgcctcgacgtcttccacggctacccgggccccaagtgcacgccatgggtcagtaagagcaactagctcagctcgaactcatcagctcagctacttgcgagtacgtatatagtacgtccacaaatgaataaaagtggctagcctacaagctaacagctgattgttcatgccgatcgacatgaacgattagcctatagctcgatcgctagctgctgctactctctttcgctcgtcactaaataactgacgaagccaactaggcctcgatcccatgtgtagagagtgtggatatgatcagctctactgaatcgttgatcatatccattctcatattttttttcgctgtttgcttcgggaaaaaaaatctgttcgctcttccaagatcgagtactcatcagctgctttgtcataagaatcaatgttaaaaaagatcgatatatattactccgtatcaacccattgtgacttctcgtgcacgctctctctcttctatatgtatgccgctgaccacaaaagtttagcaagtattttctccgtttcagatt

SEQ ID NO: 34 - protein, 181 aa, Saccharum officinarum
MGTIPVVIPAMLVVALLVVGATATATATAARPSHTDTEAIRLPSSTAAGQPWECCDWVTQDPTFRPPKWRCNDVVDKCSADCQQCEASAAGDGFVCRDWIFSLFEPPVCTPRPWDCCDLAACTRDYIPYCRCADEVESCPSNCKECELVESEESDPPRYRCLDVFHGYPGPKCTPWVSKSN

SEQ ID NO: 35 - DNA, 873 bp, Artifiicial sequence
ctttgtttcaacttcgggtacggactgtgacgcgtcccttgcattattggtgggtgcacctaacgatgcgggaagccgaactccctctataaataggaccccgtgtattcagttgcaagcacgcaacacaacgcgagcttacttctgagaagaaataagaacaatttgtgcttgaaatacaccttgtgtcaagagtgtgagtagagcgcaagatccgtgttgggaaatccgtgccgttctggaaatccgtgccgttctggtatcagagctttgtcccgggggtgaagccgaattctagaactagtggatccgcgtctcgccttctttcgtcctatacgcaatggtgaccgctcgcctccgcctcgcgctgctactactctccgtgttcctctgctccgcgtgggcgcgcccacgcctcgagccgaccatccgcctgccgtccgagcgcgccgcggcggcggccggcgacgaaacggacgacgccgtcgggacacggtgggccgtgctcgtcgccggctccagcggctactacaactaccgccaccaggcagacatctgccatgcgtaccagattatgaagaagggaggactcaaggacgaggccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcc

SEQ ID NO: 36 - protein, 178 aa, Saccharum officinarum
MVTARLRLALLLLSVFLCSAWARPRLEPTIRLPSERAAAAAGDETDDAVGTRWAVLVAGSSGYYNYRHQADICHAYQIMKKGGLKAMVSKGEELFTGVVDEPILVELDGDVNGHKFSVSGEGEGDATYGKLTLKFICTTGKLPVPWPTLVTTFTYGVQCFSRYPDHMKQHDFFKSAMP

SEQ ID NO: 37 - protein, 39 aa, Artificial sequence
  Artificial consensus sequence
MVXXRLRLALLLXXXXLCSAWARPRLEPTIRLPSERAAA

SEQ ID NO: 38 - protein, 21 aa, Saccharum officinarum
MRPAGQLLLPLLLLAVAASAA

SEQ ID NO: 39 - protein, 21 aa, Saccharum officinarum
MRPAGQLLLPLLLLAVSVAAA

SEQ ID NO: 40 - protein, 23 aa, Saccharum officinarum
MGTIPVVIPAMLVVALLVVGATA

SEQ ID NO: 41 - DNA, 356 bp, Saccharum officinarum
ttcgattatgcgtcccgccggccagctactcctcccgctcctcctcctcgccgtagccgcctccgccgccggctcttgggaggatggccatgggagcatcctccgcctcccctcctcctcgtccccgcggcggttcccccgctccgccgccgtcgacctgatccgcgcgctgaacctccacccgaccgacgcctccacgcccctctccaccgccggcgtcgagggcgccctcgcccccgcggggaccctcgtcgagaggcccatccgcctcgcgtcctttgcggacgcaggcgacgccggcacgtcggtggaggacctcggccaccacgcggggtactacaatcactagtgaattc

SEQ ID NO: 42 - protein, 113 aa, Saccharum officinarum
MRPAGQLLLPLLLLAVAASAAGSWEDGHGSILRLPSSSSPRRFPRSAAVDLIRALNLHPTDASTPLSTAGVEGALAPAGTLVERPIRLASFADAGDAGTSVEDLGHHAGYYNH

SEQ ID NO: 43 - DNA, 516 bp, Saccharum officinarum
gaattcactagtgattgcgggatccgttacagctctgcttatgaggatggggcataggacatgtggcacgatcttcatcctcctctacgtgatctctacgtcgacattgctcgcgtcatcgaacgaacatggcctcatccgtatcccgctgaagaagagatcgataatggacagcatctacggtgagcttctgccaaagccgccgagcgcgctggagaagacgaagcaagccgcggcagggccagggccagggagagaagaggccgtcggcgtcgacgacccggtgcgggacgccatcgcgcaagctcgcgagcggcagcaccagatgctggtcgaggccgcggccacggagcgcaggcgcaggtactactggagttacagcggcgggggcaagggaaacagcagccgtttgcatgagggtggcctcggccaagggaacatcgttgcgctcaagaacttcctgaatgctcagtactttgggcagattggggtcgccatggtagcacaatcgaattc

SEQ ID NO: 44 - protein, 151 aa, Saccharum officinarum
MRMGHRTCGTIFILLYVISTSTLLASSNEHGLIRIPLKKRSIMDSIYGELLPKPPSALEKTKQAAAGPGPGREEAVGVDDPVRDAIAQARERQHQMLVEAAATERRRRYYWSYSGGGKGNSSRLHEGGLGQGNIVALKNFLNAQYFGQIGV

SEQ ID NO: 45 - DNA, 54 bp, Artificial sequence
  Synthetic PCR primer
ctctgctccgcttgggctcgtggatccggagctagcaagggcgaggagctgttc

SEQ ID NO: 46 - DNA, 20 bp, Artificial sequence
  Synthetic PCR primer
gtcgtagcagataccactct

SEQ ID NO: 47 - DNA, 75 bp, Artificial sequence
  Synthetic nested primer
actagtatggtgaccgctcgcctccgcctcgcgctgctactactctccgtgttcctctgctccgcgtgggcgcgc

SEQ ID NO: 48 - DNA, 29 bp, Artificial sequence
  Synthetic PCR primer
gcgatcgatttacttgtacagctcgtcca

SEQ ID NO: 49 - DNA, 45 bp, Artificial sequence
  Synthetic PCR primer
gcgatcgatttacagctcgtccttcttgtacagctcgtccatgcc

SEQ ID NO: 50 - DNA, 60 bp, Artificial sequence
  Synthetic PCR primer
ttcctctgctccgcgtgggcgcgcccacgcctcgagccgaccatccgcctgccgtccgag

SEQ ID NO: 51 - DNA, 68 bp, Artificial sequence
  Synthetic PCR primer
ggatccgacggcgtcgtccgtttcgtcgccggccgccgccgcggcgcgctcggacggcaggcggatgg

SEQ ID NO: 52 - DNA, 2821 bp, Artificial sequence
  Expression construct plasmid
ggttgcatggaaggttggggaggagtttgtaaatggaaagaacaatcaggacaaccaagatggtcagagaagatttgtgcttatgcgagtggaaagtttaatccgatcaagagcacaattgatgcagaaattcaagcagtcatcaacagcttggataaattcaagatatattatcttgataaaaaggagttgatcatcaggacggatagtcaagcgatagtcagtttctacaagaagagtagtgaccacaaaccctcaagggtaagatggttagctttcactgactatatcactggaacaggattggatgtgaagtttgagcatattgacggcaaggataatgtgctagcagacactctgtcaaggctagtaaaaatcatatgccacaaggagaaacatccatcagaaacaatattgatcaacgttgcagaagaaatacttcagaaaggaagtattggagcaaaaagaaagttgggagaaatgataagtggatatgaagcttggatgacaagaatccaagaacacaaaatcaagacactaacacttatcgaaaaaccagtttttaaatgtggttgcaggaaacctgctaggcttcacacgtccaggacatcaagaaatccgggaagagaattttactcatgtgaaaataaagcatgtttcacttgggtatggaaggatcagattgatgaatacgttcaagaagtgatgacgtggaacgaccaagtaagccagttgccagaagaaccagaaggctacaatgaaggatgcacgattgaagacgcattcgatctgctagacgtcagcaatgacgatcaatgggcaaggtcgtaagccatgacgtagcggaagtgatggaccccataccactggatggcactaaccagtgtgacaaggatacgagatgccaagtgagctggatagcactcactttatgtaaagagtggtctgcgtaccaactccactatagtctgtctgaggtgcgatgctgtgtcacgcacaaagactttagattcctttgcgtgagatgtacgcaaagcagtgtgtccagagtgtgctgtgacgcgtcccttgcattattggtgggtgcacctaacgatgcgggaagccgaactccctctataaataggaccccgtgtattcagttgcaagcacgcaacacaacgcgagcttacttctgagaagaaataagaacaatttgtgcttgaaatacaccttgtgtcaagagtgtgagtagagcgcaagatccgtgttgggaaatccgtgccgttctggtatcagagctttgtcccgggggtgaagccgaattctagaactagtatggtgaccgctcgcctccgcctcgcgctgctactactctccgtgttcctctgctccgcttgggctcgtggatccggagctagcaagggcgaggagctgttcaccggggtggtgcccatcctggtcgagctggacggcgacgtaaacggccacaagttccgcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaatcgataccgtcgacctcgagatccggctcttcggattgctttctttggttcagtgttggatgtttccccttctctcgttgtctcctctacgcaaccgccgacagtgcagcagcgcactgaggccgtctatgttttgtttccccaagaacgcggagcttatatgttaaaaaagtatattatggacgttcgcttatttataataaaggctacccaatgtgtttgattttacctgtatccgtctgctgtgggtggaagaatcccgtaccgtagttgccaagacgtgtggtgtgggggttgaagctttgctctggcccaacctaatgttatctgggctctgtattgggccgatctcggcctacttggacgatgaaccgctgggtcaatggtcaaacaccgcgcgcatgtcttcttcttctccctcccctactaatttaatggaaatagatatatggacccccaaaaataaaaataaaaaatgtcctcctctcgcatccgacgcaccaacaaacgggccgtggcaaaaagcagtaggagaggtttcctatcgtcgtcggtcgtgaagagtagcaagtagatcgagcccaagcgaggaacgtcacgtgcgagcatcattcgaatttcgagggggggcccggtacccaattcgccctatagtgagtcgtattacgcgcgctcactggccgtgaattgaacctttgttccaactga

SEQ ID NO: 53 - DNA, 2851 bp, Artificial sequence
  Expression plasmid construct
ggttgcatggaaggttggggaggagtttgtaaatggaaagaacaatcaggacaaccaagatggtcagagaagatttgtgcttatgcgagtggaaagtttaatccgatcaagagcacaattgatgcagaaattcaagcagtcatcaacagcttggataaattcaagatatattatcttgataaaaaggagttgatcatcaggacggatagtcaagcgatagtcagtttctacaagaagagtagtgaccacaaaccctcaagggtaagatggttagctttcactgactatatcactggaacaggattggatgtgaagtttgagcatattgacggcaaggataatgtgctagcagacactctgtcaaggctagtaaaaatcatatgccacaaggagaaacatccatcagaaacaatattgatcaacgttgcagaagaaatacttcagaaaggaagtattggagcaaaaagaaagttgggagaaatgataagtggatatgaagcttggatgacaagaatccaagaacacaaaatcaagacactaacacttatcgaaaaaccagtttttaaatgtggttgcaggaaacctgctaggcttcacacgtccaggacatcaagaaatccgggaagagaattttactcatgtgaaaataaagcatgtttcacttgggtatggaaggatcagattgatgaatacgttcaagaagtgatgacgtggaacgaccaagtaagccagttgccagaagaaccagaaggctacaatgaaggatgcacgattgaagacgcattcgatctgctagacgtcagcaatgacgatcaatgggcaaggtcgtaagccatgacgtagcggaagtgatggaccccataccactggatggcactaaccagtgtgacaaggatacgagatgccaagtgagctggatagcactcactttatgtaaagagtggtctgcgtaccaactccactatagtctgtctgaggtgcgatgctgtgtcacgcacaaagactttagattcctttgcgtgagatgtacgcaaagcagtgtgtccagagtgtgctgtgacgcgtcccttgcattattggtgggtgcacctaacgatgcgggaagccgaactccctctataaataggaccccgtgtattcagttgcaagcacgcaacacaacgcgagcttacttctgagaagaaataagaacaatttgtgcttgaaatacaccttgtgtcaagagtgtgagtagagcgcaagatccgtgttgggaaatccgtgccgttctggaaatccgtgccgttctggtatcagagctttgtcccgggggtgaagccgaattctagaactagtatggtgaccgctcgcctccgcctcgcgctgctactactctccgtgttcctctgctccgcttgggctcgtggatccggagctagcaagggcgaggagctgttcaccggggtggtgcccatcctggtcgagctggacggcgacgtaaacggccacaagttccgcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcwcggcatggacgagctgtacaagaaggacgagctgtaaatcgataccgtcgacctcgagatccggctcttcggattgctttctttggttcagtgttggatgtttccccttctctcgttgtctcctctacgcaaccgccgacagtgcagcagcgcactgaggccgtctatgttttgtttccccaagaacgcggagcttatatgttaaaaaagtatattatggacgttcgcttatttataataaaggctacccaatgtgtttgattttacctgtatccgtctgctgtgggtggaagaatcccgtaccgtagttgccaagacgtgtggtgtgggggttgaagctttgctctggcccaacctaatgttatctgggctctgtattgggccgatctcggcctacttggacgatgaaccgctgggtcaatggtcaaacaccgcgcgcatgtcttcttcttctccctcccctactaatttaatggaaatagatatatggacccccaaaaataaaaataaaaaatgtcctcctctcgcatccgacgcaccaacaaacgggccgtggcaaaaagcagtaggagaggtttcctatcgtcgtcggtcgtgaagagtagcaagtagatcgagcccaagcgaggaacgtcacgtgcgagcatcattcgaatttcgagggggggcccggtacccaattcgccctatagtgagtcgtattacgcgcgctcactggccgtgaattgaacctttgttccaactga

SEQ ID NO: 54 - DNA, 2225 bp, Artificial sequence
  Expression plasmid construct
ggttgcatggaaggttggggaggagtttgtaaatggaaagaacaatcaggacaaccaagatggtcagagaagatttgtgcttatgcgagtggaaagtttaatccgatcaagagcacaattgatgcagaaattcaagcagtcatcaacagcttggataaattcaagatatattatcttgataaaaaggagttgatcatcaggacggatagtcaagcgatagtcagtttctacaagaagagtagtgaccacaaaccctcaagggtaagatggttagctttcactgactatatcactggaacaggattggatgtgaagtttgagcatattgacggcaaggataatgtgctagcagacactctgtcaaggctagtaaaaatcatatgccacaaggagaaacatccatcagaaacaatattgatcaacgttgcagaagaaatacttcagaaaggaagtattggagcaaaaagaaagttgggagaaatgataagtggatatgaagcttggatgacaagaatccaagaacacaaaatcaagacactaacacttatcgaaaaaccagtttttaaatgtggttgcaggaaacctgctaggcttcacacgtccaggacatcaagaaatccgggaagagaattttactcatgtgaaaataaagcatgtttcacttgggtatggaaggatcagattgatgaatacgttcaagaagtgatgacgtggaacgaccaagtaagccagttgccagaagaaccagaaggctacaatgaaggatgcacgattgaagacgcattcgatctgctagacgtcagcaatgacgatcaatgggcaaggtcgtaagccatgacgtagcggaagtgatggaccccataccactggatggcactaaccagtgtgacaaggatacgagatgccaagtgagctggatagcactcactttatgtaaagagtggtctgcgtaccaactccactatagtctgtctgaggtgcgatgctgtgtcacgcacaaagactttagattcctttgcgtgagatgtacgcaaagcagtgtgtccagaggtgtgctgtgacgcgtcccttgcattattggtgggtgcacctaacgatgcgggaagccgaactccctctataaataggaccccgtgtattcagttgcaagcacgcaacacaacgcgagcttacttctgagaagaaataagaacaatttgtgcttgaaatacaccttgtgtcaagagtgtgagtagagcgcaagatccgtgttgggaaatccgtgccgttctggaaatccgtgccgttctggtatcagagcttgtatactagtatggtgaccgctcgcctccgcatcgcgctgctactactctccgtgttcctctgctccgcttgggctcgtccacgtcttgaacctactatccgcctgccgtccbamhgaacgtgctgctgcagctgctggtgatgaaactgacgatgccgtcggatccggaggttcaggtggagctagcaagggcgaggagctgttcaccggggtggtgcccatcctggtcgagctggacggcgacgtaaacggccacaagttccgcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaagcggccgctgcgaggagtctggt

SEQ ID NO: 55 - DNA, 2159 bp, Artificial sequence
  Expression construct plasmid
ggttgcatggaaggttggggaggagtttgtaaatggaaagaacaatcaggacaaccaagatggtcagagaagatttgtgcttatgcgagtggaaagtttaatccgatcaagagcacaattgatgcagaaattcaagcagtcatcaacagcttggataaattcaagatatattatcttgataaaaaggagttgatcatcaggacggatagtcaagcgatagtcagtttctacaagaagagtagtgaccacaaaccctcaagggtaagatggttagctttcactgactatatcactggaacaggattggatgtgaagtttgagcatattgacggcaaggataatgtgctagcagacactctgtcaaggctagtaaaaatcatatgccacaaggagaaacatccatcagaaacaatattgatcaacgttgcagaagaaatacttcagaaaggaagtattggagcaaaaagaaagttgggagaaatgataagtggatatgaagcttggatgacaagaatccaagaacacaaaatcaagacactaacacttatcgaaaaaccagtttttaaatgtggttgcaggaaacctgctaggcttcacacgtccaggacatcaagaaatccgggaagagaattttactcatgtgaaaataaagcatgtttcacttgggtatggaaggatcagattgatgaatacgttcaagaagtgatgacgtggaacgaccaagtaagccagttgccagaagaaccagaaggctacaatgaaggatgcacgattgaagacgcattcgatctgctagacgtcagcaatgacgatcaatgggcaaggtcgtaagccawgacgtagcggaagtgatggaccccataccactggatggcactaaccagtgtgacaaggatacgagatgccaagtgagctggatagcactcactttatgtaaagagtggtctgcgtaccaactccactatagtctgtctgaggtgcgatgctgtgtcacgcacaaagactttagattcctttgcgtgagatgtacgcaaagcagtgtgtccagaggtgtgctgtgacgcgtcccttgcattattggtgggtgcacctaacgatgcgggaagccgaactccctctataaataggaccccgtgtattcagttgcaagcacgcaacacaacgcgagcttacttctgagaagaaataagaacaatttgtgcttgaaatacaccttgtgtcaagagtgtgagtagagcgcaagatccgtgttgggaaatccgtgccgttctggaaatccgtgccgttctggtatcagagctttgtatactagtatggtgaccgctcgcctccgcctcgcgctgctactactctccgtgttcctctgctccgcttgggctcgtccacgtcttgaacctactatccgcctgccgtccgaacgcggatccggaggttcaggtggagctagcaagggcgaggagctgttcaccggggtggtgcccatcctggtcgagctggacggcgacgtaaacggccacaagttccgcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccrggatcactctcggcatggacgagctgtacaagtaa

SEQ ID NO: 56 - DNA, 2134 bp, Artificial sequence
  Expression construct plasmid
ggttgcatggaaggttggggaggagtttgtaaatggaaagaacaatcaggacaaccaagatggtcagagaagatttgtgcttatgcgagtggaaagtttaatccgatcaagagcacaattgatgcagaaattcaagcagtcatcaacagcttggataaattcaagatatattatcttgataaaaaggagttgatcatcaggacggatagtcaagcgatagtcagtttctacaagaagagtagtgaccacaaaccctcaagggtaagatggttagctttcactgactatatcactggaacaggattggatgtgaagtttgagcatattgacggcaaggataatgtgctagcagacactctgtcaaggctagtaaaaatcatatgccacaaggagaaacatccatcagaaacaatattgatcaacgttgcagaagaaatacttcagaaaggaagtattggagcaaaaagaaagttgggagaaatgataagtggatatgaagcttggatgacaagaatccaagaacacaaaatcaagacactaacacttatcgaaaaaccagtttttaaatgtggttgcaggaaacctgctaggcttcacacgtccaggacatcaagaaatccgggaagagaattttactcatgtgaaaataaagcatgtttcacttgggtatggaaggatcagattgatgaatacgttcaagaagtgatgacgtggaacgaccaagtaagccagttgccagaagaaccagaaggctacaatgaaggatgcacgattgaagacgcattcgatctgctagacgtcagcaatgacgatcaatgggcaaggtcgtaagccatgacgtagcggaagtgatggaccccataccactggatggcactaaccagtgtgacaaggatacgagatgccaagtgagctggatagcactcactttatgtaaagagtggtctgcgtaccaactccactatagtctgtctgaggtgcgatgctgtgtcacgcacaaagactttagattcctttgcgtgagatgtacgcaaagcagtgtgtccagagtgtgctgtgacgcgtcccttgcattattggtgggtgcacctaacgatgcgggaagccgaactccctctataaataggmcccsgtgtattcagttgcaagcacgcaacacaacgcgagcttacttctgagaagaaataagaacaatttgtgcttgaaatacaccttgtgtcaagagtgtgagtagagcgcaagatccgtgttgggaaatccgtgccgttctggaaatccgtgccgttctggtatcagagctttgtatactagtatggtgaccgctcgcctccgcctcgcgctgctactactctccgtgttcctctgctccgcttgggctcgtatccgcctgccgtccggatccggaggttcaggtggagctagcaagggcgaggagctgttcaccggggtggtgcccatcctggtcgagctggacggcgacgtaaacggccacaagttccgcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 57 - protein, 7 aa, Artificial sequence
  Synthetic peptide linker
GGSGGAS

SEQ ID NO: 58 - DNA, 69 bp, Artificial sequence
  Synthetic primer
ggatccgcgctcggacggcaggcggatggtcggctcgaggcgtgggcgcgcccacgcggagcagaggaa

SEQ ID NO: 59 - DNA, 45 bp, Artificial sequence
  Synthetic primer
ggatccggacggcaggcggatgcgcgcccacgcggagcagaggaa

SEQ ID NO: 60 - DNA, 27 bp, Artificial sequence
  Synthetic PCR primer
ggatccgacggcgtcgtccgtttcgtc

SEQ ID NO: 61 - protein, 264 aa, Artificial sequence
  Synthetic GFP construct
MVTARLRLALLLLSVFLCSAWARGSGASKGEELFTGVVPILVELDGDVNGHKFRVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 62 - protein, 268 aa, Artificial sequence
  Synthetic GFP construct
MVTARLRLALLLLSVFLCSAWARGSGASKGEELFTGVVPILVELDGDVNGHKFRVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITXGMDELYKKDEL

SEQ ID NO: 63 - protein, 294 aa, Artificial sequence
  Synthetic GFP construct
MVTARLRIALLLLSVFLCSAWARPRLEPTIRLPSERAAAAAGDETDDAVGSGGSGGASKGEELFTGVVPILVELDGDVNGHKFRVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 64 - protein, 281 aa, Artificial sequence
  Synthetic GFP construct
MVTARLRLALLLLSVFLCSAWARPRLEPTIRLPSERGSGGSGGASKGEELFTGVVPILVELDGDVNGHKFRVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAXITLGMDELYK

SEQ ID NO: 65 - protein, 273 aa, Artificial sequence
  Synthetic GFP construct
MVTARLRLALLLLSVFLCSAWARIRLPSGSGGSGGASKGEELFTGVVPILVELDGDVNGHKFRVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 66 - protein, 4 aa, Saccharum officinarum
IRLP

SEQ ID NO: 67 - protein, 4 aa, Saccharum officinarum
RLPS

(19) United States
(12) Patent Application Publication
(10) Pub. No.: US 2009/0070895 A1
(43) Pub. Date: Mar. 12, 2009

(54) VACUOLE TARGETING PEPTIDE AND NUCLEIC ACID

(76) Inventors: Anne Rae, Queensland (AU); Roseanne Casu, Queensland (AU); Mark Jackson, Queensland (AU); Christopher Grof, Queensland (AU)

Correspondence Address: ROSENBAUM & ASSOCIATES, P.C., 650 DUNDEE ROAD, SUITE #380, NORTHBROOK, IL, 60062, US

(21) Appl. No.: 11/722,654

(22) PCT Filed: Dec. 23, 2005
(86) PCT No.: PCT/AU05/01970

§ 371 Date: Jun. 22, 2007

(30) Foreign Application Priority Data
Dec. 24, 2004 (AU) 200407342

(51) Int. Cl. C12N 15/82; C07K 14/00; C12N 9/00; C12N 9/90; C12N 9/10; A01H 5/00; C12P 21/04; C12N 5/04; C12N 9/14; C12N 9/02; C12N 15/11; C12N 15/00

(52) U.S. Cl. 800/279; 530/350; 435/183; 435/233; 435/193; 435/195; 435/189; 536/23.4; 435/320.1; 800/298; 800/320; 800/320.1; 800/320.2; 800/320.3; 435/419; 435/317.1; 435/69.1

(57) ABSTRACT

The present invention relates to a plant vacuole targeting sequence X₁X₂X₃PX₄ wherein X₁ is a hydrophobic amino acid, X₂ is a basic amino acid, X₃ is a hydrophobic amino acid, P is proline; and X₄ is a hydrophilic amino acid, such as the sequences IRLPS, IKLPS, LRLPS and LKLPS. The vacuole targeting sequence may be present in a chimeric protein linked to an amino acid sequence of a heterologous protein to facilitate vacuole vacuole targeting of the expressed chimeric protein in a plant cell. The invention is applicable to production of expressed, chimeric proteins in monocots and dicots, and in particular monocots such as cereals and sugarcane.

### FIELD OF THE INVENTION

THIS INVENTION relates to an isolated vacuole targeting peptide and nucleic acid encoding the isolated vacuole targeting peptide. This invention further relates to nucleic acid constructs comprising the isolated nucleic acid for expressing proteins that are specifically targeted to a vacuole of a plant.

### BACKGROUND OF THE INVENTION

Plant cells may comprise a number of different vacuoles, which can be distinguished by a presence of specific marker proteins. Major classes of vacuoles include the protein storage vacuole, which is typically found in seeds, and the lytic vacuole which is characterised by low pH and proteolytic activity (Bassham and Raikhel 2000). Proteins are targeted to vacuoles via a secretory endomembrane system and vesicle trafficking. The destination of proteins within the endomembrane system is determined by short peptide sequences which may be located within the protein or at the amino- (N-) or carboxy- (C-) terminus. Proteins that possess the secretory signal peptide, but lack further targeting sequences are generally secreted (Bassham and Raikhel 2000). A number of peptide sequences that direct proteins to vacuoles have been characterised (Vitale and Raikhel 1999). Targeting to the lytic vacuole may be associated with propeptides located at the N-terminus of a protein. The best characterised of these peptides are from sweet potato sporamin and barley aleurain, which both comprise a peptide having an amino acid sequence “-NPIR-”. While these peptides have been used successfully in some heterologous systems, they are of limited use as they are not universally functional in targeting introduced proteins into the lytic vacuole.

n mature sugarcane stems, the vacuole occupies a large volume of the storage parenchyma cells (Jacobsen et al, 1992). Because of their large size and location in a storage tissue, these vacuoles have been regarded as an ideal site for the production and storage of commercially valuable products in transgenic sugarcane. However, targeting peptides that are functional in sugarcane have not yet been identified. The “NPIR-like” N-terminal propeptide from sweet potato sporamin and the C-terminal propeptide from chitinase were tested for their ability to direct a number of reporter genes into the vacuole of sugarcane cells.

The sporamin sequence was also investigated in International Publication WO2004/035750 as a source of potential vacuole targeting sequences. However, there was considerable variability in the vacuole targeting ability of the sequences tested.

Overall, the sweet potato sporamin sequence has proven to be an unpredictable source of potential vacuole targeting sequences.

### SUMMARY OF THE INVENTION

The present invention seeks to overcome or alleviate the inability of prior art targeting sequences to specifically target expressed proteins to a plant vacuole.

With this in mind, the present invention is directed to a plant vacuole targeting sequence that has an advantage of being specific and/or universal, in that the targeting sequence may be useful in targeting expressed proteins specifically to the plant vacuole in a wide variety of plants.

In a broad form, the invention provides a vacuole targeting sequence X₁X₂X₃PX₄ (SEQ ID NO:1) wherein:

X₁ is a hydrophobic amino acid;

X₂ is a basic amino acid;

X₃ is a hydrophobic amino acid

P is proline; and

X₄ is a hydrophilic amino acid.

Preferably, the vacuole targeting sequence is (I/L)(R/K)LPS (SEQ ID NO:24).

In particular embodiments of this broad form, the vacuole targeting sequence comprises an amino acid sequence IRLPS (SEQ ID NO: 2), IKLPS (SEQ ID NO: 3), LRLPS (SEQ ID NO: 4) or LKLPS (SEQ ID NO: 5).

In a first aspect, the invention provides an isolated protein comprising said vacuole targeting sequence.

Preferably, the isolated protein is a chimeric protein that further comprises an amino acid sequence of a heterologous protein.

Preferably, said heterologous protein does not normally comprise said vacuole targeting sequence or normally comprises a different vacuole targeting sequence.

Suitably, the vacuole targeting sequence and the amino acid sequence of the heterologous protein are arranged so that said vacuole targeting sequence is capable of facilitating targeting of the chimeric protein to a vacuole in a plant cell.

While the vacuole targeting sequence of the invention is set forth herein as a five (5) residue sequence, the vacuole targeting sequence may be provided within the context of additional flanking sequence, inclusive of a secretory signal peptide sequence.

Preferably, the additional flanking sequences are present at an amino terminal end of a sequence, such as shown in FIGS. 1-9.

A secretory signal peptide is well known in the art and is capable of directing a protein to an endomembrane system of a cell. Examples of preferred secretory signal peptides are shown in FIGS. 1, 2, 3, 4, 5, 6 and 8.

Preferably, the secretory signal peptide comprises an amino acid sequence selected from the group consisting of:

### MVTARLRLALLLLSVFLCSAWA (SEQ ID NO: 9); MRPAGQLLLPLLLLAVAASM (SEQ ID NO: 38); MRPAGQLLLPLLLLAVSVAAA (SEQ ID NO: 39); and MGTIPWIPAMLWALLVVGATA (SEQ ID NO: 40).

Preferably, the heterologous protein is selected from the group consisting of: a sucrose modifying enzyme, a hexose modifying enzyme, a protein capable of use as an industrial enzyme, a protein capable of use as a pharmaceutical composition and/or diagnostic reagent, a protein capable of use in crop protection, a protein characterized by culinary or industrial properties and a vacuolar metabolite modifying enzyme.

Preferably, the sucrose modifying enzyme comprises sucrose isomerase, fructosyl transferases, invertase, amylosucrase, dextransucrase and glucan sucrase.

Preferably, hexose modifying enzyme is capable of directly modifying a hexose structure.

More preferably, the hexose modifying enzyme comprises polyol dehydrogenase, dextran synthases and other transferase proteins.

Preferably, the protein capable of use as an industrial enzyme comprises lipases, cellulase, pectinase, hemicellulase, peroxidases, amylase, dextranase, protease, polysaccharases, lytic enzymes and other proteins.

Preferably, the protein capable of use in a pharmaceutical composition and/or diagnostic reagent comprises antigens, antibodies, antibody fragments, cytotoxic agents, anticancer proteins, immunotherapeutic agents, vaccines, hormones, cytokines and the like.

Preferably, the protein capable of use in crop protection comprises an antifungal protein, antibacterial proteins, anti-insect proteins and anti-nematode proteins.

More preferably, antifungal protein comprises plant defensins, the antibacterial protein comprises thionins, the anti-insect protein comprises Bt, protease inhibitors and avidin and the anti-nematode protein comprises collagenase.

Preferably, the protein characterized by culinary or industrial properties comprises coagulants, gelling proteins, sweet proteins, sour proteins and adhesive proteins.

Preferably, the vacuolar metabolite modifying enzyme comprises an enzyme capable of modifying a compound selected from the group consisting of a phenolic compound, tannin compound, flavonoid compound and other secondary metabolites.

Preferably, the vacuole is a lytic vacuole.

The vacuole may be of a monocotyledon plant or dicotyledon plant.

Preferably, the vacuole is of a monocotyledon.

More preferably, the monocotyledon is sugarcane, maize, wheat, barley, sorghum, rye, oats or rice.

In a second aspect, the invention provides an isolated nucleic acid encoding the isolated protein of the first aspect.

In a third aspect, the invention provides a genetic construct comprising an isolated nucleic acid encoding the vacuole targeting sequence set forth in SEQ ID NO;1 or the isolated protein of the first aspect.

Preferably, the genetic construct is an expression construct wherein the isolated nucleic acid is a transcribable nucleic acid.

Preferably, the expression construct comprises one or more regulatory elements operably linked or connected to the isolated nucleic acid to facilitate transcription thereof.

In a fourth aspect, the invention provides a method of producing a genetically-modified plant including the step of introducing the isolated nucleic acid of the second aspect or the genetic construct of the third aspect to a plant cell or tissue.

Preferably, the method includes the step of selectively propagating a genetically-transformed plant from said a plant cell or tissue.

Preferably, the plant cell or tissue is a callus.

In a fifth aspect, the invention provides a genetically-modified plant comprising the isolated nucleic acid of the second aspect or the genetic construct of the third aspect

In an sixth aspect, the invention provides a plant tissue, cell, organelle or other part obtainable from the genetically-modified plant of the fifth aspect.

Preferably, the organelle is a vacuole.

More preferably, the vacuole is a lytic vacuole.

Preferably, the plant tissue, cell, organelle or other part is selected from fruit, leaf, root, shoot, stem, flower, seed, cutting and other reproductive material useful in sexual or asexual propagation, progeny plants inclusive of F1 hybrids, male-sterile plants and all other plants and plant products derivable from the genetically-modified plant.

In a seventh aspect, the invention provides a method for producing a recombinant protein in a plant including the steps of:
(1) expressing a recombinant protein of the first aspect in a plant; and
(2) isolating the recombinant protein from a tissue, cell or organelle of said plant.

Preferably, the recombinant protein is isolated, purified or otherwise obtained from an organelle of said plant.

Preferably, the organelle is a vacuole.

More preferably, the vacuole is a lytic vacuole.

In an eighth aspect, the invention provides a method for tissue specific expression of a protein in a plant including the steps of expressing the isolated nucleic acid of the second aspect in a plant.

Preferably, a recombinant protein encoded by the isolated nucleic acid is targeted to a vacuole.

Preferably, the vacuole is a lytic vacuole.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES

In order that the invention may be readily understood and put into practical effect, preferred embodiments will now be described by way of example with reference to the accompanying figures wherein like reference numerals refer to like parts and wherein:

FIG. 1 shows a predicted amino acid sequence of sugarcane asparaginyl endopeptidase (SEQ ID NO:10). A putative signal peptide is italicized. Predicted N-terminal and C-terminal propeptides are underlined. The putative vacuolar targeting sequence is bolded and boxed.

FIG. 2 shows a nucleotide sequence (SEQ ID NO:30) of the coding region of a transcript corresponding to sugarcane asparaginyl endopeptidase and its associated predicted amino acid sequence (SEQ ID NO:9). A putative signal peptide is italicized. Predicted N-terminal and C-terminal-propeptides are underlined. The putative vacuolar targeting sequence is bolded and double-underlined.

FIG. 3 shows an amino acid sequence alignment of sugarcane asparaginyl endopeptidase with related proteins from other species. Sc, sugarcane asparaginyl endopeptidase (SEQ ID NO: 10); Zm, Zea mays C13 endopeptidase NP1 precursor (Genpept accession number AAD04883) (SEQ ID NO: 11); Os, Oryza sativa asparaginyl endopeptidase (Genpept accession number NP_{—}918390) (SEQ ID NO: 12); At, Arabidopsis thaliana vacuolar processing enzyme, gamma-isozyme precursor (SwissProt accession number VPEG_ARATH) (SEQ ID NO: 13); Nt, Nicotiana tabacum vacuolar processing enzyme-1b (Genpept accession number BAC54828) (SEQ ID NO: 14); Cs; Citrus sinensis vacuolar processing enzyme precursor (SwissProt accession number VPE_CITSI) (SEQ ID NO: 15); XI, Xenopus laevis MGC64351 protein (Genpept accession number AAH56842) (SEQ ID NO: 16); Rn, Rattus norvegicus legumain (Genpept accession number NP_{—}071562) (SEQ ID NO: 17); Bt, Bos taurus legumain (Genpept accession number NP_{—}776526) (SEQ ID NO: 18); Hs, Homo sapiens legumain precursor (SwissProt accession number LGMN_HUMAN) (SEQ ID NO: 18), identical and similar amino acids are boxed.

FIG. 4 shows location of a putative vacuolar targeting sequence in four sugarcane proteins, asparaginyl endopeptidase (SEQ ID NO: 10), carboxypeptidase (SEQ ID NO: 20), predicted trypsin inhibitor protein (SEQ ID NO: 21) and aspartic protease (SEQ ID NO: 22), which all comprise a predicted secretory signal peptide, but are not otherwise related, the putative vacuolar targeting motif is underlined, stars mark predicted peptide cleavage sites.

FIG. 5 shows a predicted nucleotide sequence (SEQ ID NO: 31) and deduced amino acid sequence (SEQ ID NO: 32) of TC57738, a sugarcane consensus DNA sequence homologous to carboxypeptidase as shown in FIG. 4 derived from nucleic acid fragments, a putative signal peptide is italicized and underlined, a putative vacuolar targeting sequence is bolded and double underlined, this sequence appears to be prematurely terminated, possibly due to sequence anomalies in the ESTs used to prepare the consensus sequence.

FIG. 6 shows a partial nucleotide sequence (SEQ ID NO: 41) and deduced amino acid sequence (SEQ ID NO: 42) of a sugarcane carboxypeptidase cloned into pGemT easy vector (Promega), a putative signal peptide is italicized and underlined, a putative vacuolar targeting sequence is bolded and double underlined.

FIG. 7 shows a partial nucleotide sequence (SEQ ID NO: 43) and deduced amino acid sequence (SEQ ID NO: 44) of a sugarcane aspartic protease nucleic acid cloned into PgemT easy vector (Promega), a putative vacuolar targeting sequence is bolded and double underlined.

FIG. 8 shows a nucleotide sequence (SEQ ID NO: 33) and deduced amino acid sequence (SEQ ID NO: 34) of TC50252, a sugarcane consensus DNA sequence homologous to trypsin inhibitor as shown in FIG. 4, a putative signal peptide is italicized and underlined, a putative vacuolar targeting sequence is bolded and double underlined.

FIG. 9 shows a partial nucleotide sequence (SEQ ID NO: 35) and amino acid sequence (SEQ ID NO: 36) of the pEndoNTPP-GFP expression construct comprising nucleotides encoding a secretory signal peptide, a putative vacuolar targeting motif and a first 40 amino acids of a mature protein for sugarcane endopeptidase (underlined), linked in-frame to a nucleic acid comprising an nucleotide sequence for green fluorescent protein (GFP) (dotted underlined), the putative vacuolar targeting motif is bolded and double underlined, a restriction site NcoI, that links the two nucleic acids is bolded and italicized.

FIG. 10A shows control cells transformed with pCvGFPT without the addition of a secretory signal peptide or vacuole targeting peptide, GFP is visible in peripheral cytoplasm and in the nucleus.

FIG. 10B shows cells transformed with pCvGFPT comprising a putative targeting domain from the endopeptidase gene (i.e. pEndoNTPP-GFP as shown in FIG. 9), GFP is visible in a central vacuole and absent from nucleus and peripheral cytoplasm, a yellow sphere is an inclusion comprising phenolic compounds, which is characteristic of a vacuole in sugarcane.

FIG. 10C shows cells incubated with a vacuolar lumen marker dye, CellTracker Blue CMAC, the dye accumulated in a central vacuole, while the nucleus and the peripheral cytoplasm remained relatively dark, some autofluorescence of the cell wall is also visible.

FIG. 10D shows double labeling of the same cell in FIG. 10C with a tonoplast marker, MDY-64 showing that the compartment accumulating the CellTracker dye is delimited by the tonoplast, confirming that this structure is a vacuole.

FIG. 11 shows nucleotide sequence of a gfp expression construct designed to localise gfp to the apoplastic space (pCVsgfp; SEQ ID NO:51). The signal peptide (italicised) of the sugarcane asparaginyl endopeptidase gene (ScVPE-1) was fused in frame with the reporter gene GFP. A small linker was included between the predicted signal peptide cleavage site and the start of gfp. The gfp amino acid sequence is indicated in non-italicized single letter code.

FIG. 12 shows a nucleotide sequence of a gfp expression construct designed to localise gfp to the endoplasmic reticulum (pCvsgfpKDEL; SEQ ID NO:52). The signal peptide (italicised) of the sugarcane asparaginyl endopeptidase gene (ScVPE-1) was fused in frame with the reporter gene GFP. A small linker was included between the predicted signal peptide cleavage site and the start of gfp. A KDEL motif was added to the C terminus for retention of gfp in the endoplasmic reticulum. The gfp amino acid sequence is indicated in non-italicized single letter code.

FIG. 13 shows a nucleotide sequence of a gfp expression construct containing the complete NTPP of a sugarcane asparaginyl endopeptidase gene (ScVPE-1) fused in frame with the reporter gene GFP (pCvEndoExp1-gfp; SEQ ID NO:53). A small amino acid linker was included between the end of the endopeptidase NTPP and the start of gfp to ensure flexibility of the protein fusion. Italicised is a predicted signal peptide. A putative vacuolar targeting motif is bolded and double underlined. The gfp amino acid sequence is indicated in non-italicized, non-underlined single letter code without bolding.

FIG. 14 shows a nucleotide sequence of a gfp expression construct containing a partial region of the NTPP of a sugarcane asparaginyl endopeptidase gene (ScVPE-1) fused in frame with the reporter gene GFP (pCvEndoExp2-gfp; SEQ ID NO: 54). An 8 amino acid linker was included between the end of the endopeptidase sequence and the start of gfp to ensure flexibility of the protein fusion. Italicised is a predicted signal peptide. A putative vacuolar targeting motif is bolded and double underlined. The gfp amino acid sequence is indicated in non-italicized, non-underlined single letter code without bolding.

FIG. 15 shows a nucleotide sequence of a gfp expression construct containing a partial region of the NTPP of a sugarcane asparaginyl endopeptidase gene (ScVPE-1) fused in frame with the reporter gene GFP (pCvEndoExp3-gfp; SEQ ID NO: 55). An 8 amino acid linker was included between the end of the endopeptidase sequence and the start of gfp to ensure flexibility of the protein fusion. Italicised is a predicted signal peptide. A putative vacuolar targeting motif is bolded and double underlined. The gfp amino acid sequence is indicated in non-italicized, non-underlined single letter code without bolding.

### DETAILED DESCRIPTION OF THE INVENTION

Unless defined otherwise, all technical and scientific terms used herein have a meaning as commonly understood by those of ordinary skill in the art to which the invention belongs. Although any method and material similar or equivalent to those described herein can be used in the practice or testing of the present invention, preferred methods and materials are described. For the purpose of the present invention, the following terms are defined hereinafter.

The present invention relates to identification of an N-terminal propeptide (NTPP) from a sugarcane protein that is effective in directing a fusion protein, exemplified by a reporter protein, into a vacuole in sugarcane. Within this propeptide, is a short peptide sequence motif that is highly conserved amongst proteases of the legumain family from a range of different species. This is significant because the proteins of this family are almost entirely located within the vacuole. In addition, the same motif is present in the sequences of three other proteins from sugarcane that are predicted to be located in the vacuole, but which are otherwise unrelated. Because of the strong association between vacuolar localization and the presence of this motif, it is proposed that a vacuolar targeting peptide comprises the motif, X₁X₂X₃PX₄ wherein X₁ and X₁ are a hydrophobic amino acid; X₂ is a basic amino acid, P is proline and X₄ is a hydrophilic amino acid.

The vacuolar targeting sequence of the invention may have applications in targeting a heterologous protein of interest, including novel synthetic proteins, preferably commercially valuable proteins such as enzymes and other proteins described herein, to the vacuole in transgenic sugarcane. Several properties of the vacuole make it an attractive location for expressing exogenous proteins, including enzymes. In mature stem parenchyma cells, the vacuole is large and abundantly supplied with sucrose as a potential carbon supply. Furthermore, an ability to compartmentalize an expressed protein away from a majority of cellular metabolism minimizes potential detrimental effects of the expressed protein. The presence of the targeting motif in endopeptidases from plants other than sugarcane suggests that it may be effective in a wide range of crop plants.

When combined with tissue-specific and/or conditional promoters, the vacuolar targeting motif of the present invention may provide a means for tight control of transgene expression and subcellular localization.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material includes material in native and recombinant form.

By “protein” is meant an amino acid polymer, comprising natural and/or non-natural amino acids, including L- and D-isomeric forms, as are well understood in the art.

Typically, the term “peptide” refers to a protein having not more than fifty (50) contiguous amino acids.

Typically, the term “polypeptide” refers to a protein having more than fifty (50) contiguous amino acids.

By “endogenous” nucleic acid, protein, peptide or polypeptide is meant a nucleic acid, protein, peptide or polypeptide that may be normally found in a native or non-transformed cell, tissue or animal in isolation or otherwise.

By “exogenous” nucleic acid, protein, peptide or polypeptide is meant a nucleic acid, protein, peptide or polypeptide that is not normally found in a native cell, tissue or animal in isolation or otherwise. The term “exogenous” may in one preferred form describe a “transgene”.

The term “native” nucleic acid or protein also refers to “wild-type” nucleic acid or protein, which are normally obtainable from a selected organism or part thereof.

The term “non-native” nucleic acid or protein refers to a nucleic acid or protein not normally obtainable from a selected organism or part thereof. For example, a non-native protein preferably comprises a chimeric protein that may comprise two peptides or proteins not normally associated with each other as a contiguous protein and accordingly comprise non-native proteins. Likewise, a chimeric nucleic acid may comprise two or more non-native nucleic acids.

A “chimeric” gene, nucleic acid, protein, peptide or polypeptide is meant a gene, nucleic acid, protein, peptide or polypeptide that comprises two or more nucleic acid or proteins not normally associated together. Preferably the chimera comprises (i) a vacuole targeting sequence of the invention and (ii) an amino acid sequence of a heterologous protein which does not normally comprise said vacuole targeting sequence or which normally comprises a different vacuole targeting sequence.

Suitably, (i) and (ii) are arranged so that said vacuole targeting sequence is capable of facilitate targeting of the chimeric protein to a vacuole in a plant cell. Preferably, the two or more nucleic acids or proteins are not normally contiguous.

### Vacuole Targeting Sequences and Chimeric Proteins

In particular aspects, the invention provides a vacuolar targeting peptide or an isolated protein comprising same typically in the form of a chimeric protein.

In a broad form, the vacuole targeting sequence is X₁X₂X₃PX₄ wherein:

X₁ is a hydrophobic amino acid;

X₂ is a basic amino acid;

X₃ is a hydrophobic amino acid

P is proline; and

X₄ is a hydrophilic amino acid.

Preferably the motif is (I/L)(R/K)LPS (SEQ ID NO:24).

In particular embodiments of this broad form, the vacuole targeting sequence comprises an amino acid sequence IRLPS (SEQ ID NO: 2), IKLPS (SEQ ID NO: 3), LRLPS (SEQ ID NO: 4) or LKLPS (SEQ ID NO: 5).

In one particular embodiment, the vacuole targeting sequence is IRLPS (SEQ ID NO:2).

A particular feature of the present invention is that the five (5) amino acid sequence defined by SEQ ID NOS:1-5 and SEQ ID NO:24 is sufficient to effectively target proteins to a plant vacuole.

It will also be appreciated that a minimal vacuole targeting motif may consist of an amino acid sequence: IRLP, IRL, LPS or RLPS.

It will be appreciated that the consensus amino acid sequence of the vacuolar targeting peptide of the invention has been obtained, derived or otherwise deduced from sugarcane proteins as described herein, including asparaginyl endopeptidase, carboxypeptidase, trypsin inhibitor protein and aspartic protease.

Thus, while the five (5) amino acid sequence described herein is sufficient, the vacuolar targeting sequence may nevertheless be that of a peptide or polypeptide comprising additional, flanking amino acids, and thus may be up to 300 amino acids in length, or preferably comprising 250, 200, 150, 100, 90, 88, 87, 80, 70, 60, 50, 40, 30, 25, 23, 20, 15, 10, 9, 8, 7, 6, or 5 amino acids.

In a preferred embodiment, the vacuolar targeting sequence consists of the five (5) amino acid peptide motif SEQ ID NO:1-5, or SEQ ID NO:24.

In another, less preferred embodiment the vacuolar targeting sequence consists essentially of the peptide sequence defined by the five (5) amino acid peptide sequence of SEQ ID NO:1-5, or SEQ ID NO:24.

The term “consisting essentially of” or “consists essentially of” is understood to mean that there may be one, two or three additional amino acid(s) located at either or both amino and/or carboxyl end of the peptide sequence. The additional amino acids may be the same amino acids that naturally flank the vacuole targeting sequence or may be other amino acids that do not naturally flank the sequence.

Thus the vacuolar targeting peptide may be present in the form of a fragment of a sugarcane protein as herein described.

For example, a fragment may in a preferred form comprise less than 99%, 95%, 90%, 85%, 80%, 75%, 70%, 65%, 60%, 55%, 50%, 40%, 30%, 20% and even less than 10% of the entire protein.

A fragment may include a vacuole targeting sequence IRLPS (SEQ ID NO: 2), IKLPS (SEQ ID NO: 3), LRLPS (SEQ ID NO: 4) or LKLPS (SEQ ID NO: 5); a secretory signal peptide such as MVTARLRLALLLLSVFLCSAWA (SEQ ID NO: 9), MRPAGQLLLPLLLLAVAASM (SEQ ID NO: 38); MRPAGQLLLPLLLLAVSVAAA (SEQ ID NO: 39) or MGTIPVVIPAMLVVALLWGATA (SEQ ID NO: 40), a propeptide such as WARPRLEPTIRLPSERAAAAAGDETDD (SEQ ID NO: 23) or EARKELLEVMSHRSHVDNSVELIGSLLFGSEDGPRVLKAVRAAGEPLVDDWSCL KSMVRTFEAQCGSLAQYGMKHMRTFANICNAGILPEAVSKVAAQACTSIPSNP WSSIDKGFSA (SEQ ID NO: 25), MVTARLRLALLLLSVFLCSAWARPRLEPTIRLPSERAAAAAGDETDDAVGTRWA VLVAGSSGYYNYRHQADICHAYQIMKKGGLKDEN (SEQ ID NO: 6); LCSAWARPRLEPTIRLPSERAAA (SEQ ID NO: 7); or RPRLEPTIRLPSERAAAAAGDETDD (SEQ ID NO: 8).

The fragment may be a “biologically active fragment” which retains biological activity of a given protein.

For example, a biologically active fragment of asparaginyl endopeptidase, carboxypeptidase, trypsin inhibitor protein and aspartic protease may retain enzymatic activity.

A biologically active fragment, for example, may comprise a vacuole targeting sequence as hereinbefore described; a secretory signal peptide preferably comprising amino acids MVTARLRLALLLLSVFLCSAWA (SEQ ID NO: 9), MRPAGQLLLPLLLLAVAASAA (SEQ ID NO: 38); MRPAGQLLLPLLLLAVSVAAA (SEQ ID NO: 39) or MGTIPWIPAMLVVALLWGATA (SEQ ID NO: 40), or a propeptide such as WARPRLEPTIRLPSERAAMAGDETDD (SEQ ID NO: 23) and EARKELLEVMSHRSHVDNSVELIGSLLFGSEDGPRVLKAVRAAGEPLVDDWSCL KSMVRTFEAQCGSLAQYGMKHMRTFANICNAGILPEAVSKVAAQACTSIPSNP WSSIDKGFSA (SEQ ID NO: 25).

A biologically active fragment preferably constitutes at least greater than 10% of the biological activity of the entire polypeptide or peptide, preferably greater than 15% or 20%, more preferably greater than 25%, 35%, 45% and even more preferably greater than 50%, 60%, 70%, 80%, 90% and even 95% or 99% biological activity of the entire protein. The biologically activity of the biologically active fragment maybe greater than 100% of a full-length protein, for example, if an inhibitory domain is deleted.

In another embodiment, a “fragment” is a small peptide, for example of at least five, preferably at least 10 and more preferably at least 20 amino acids in length, which comprises one or more antigenic determinants or epitopes capable of being bound by an antibody.

Larger fragments comprising more than one peptide are also contemplated, and may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with a suitable proteinases. The digested fragments can be purified by, for example, high performance liquid chromatographic (HPLC) techniques.

The invention also extends to protein homologs, orthologs, variants and derivatives.

As used herein, “variant” proteins are proteins wherein one or more amino acids have been replaced by different amino acids. A variant protein includes a protein with one or several amino acid deletion, substitution and/or addition. It is well understood in the art that some amino acids may be changed to others with broadly similar properties without changing the nature of the activity of the protein (e.g. conservative substitutions).

Substantial changes in function are made by selecting substitutions that are less conservative or non-conservative as is known in the art. Generally, the substitutions which are likely to produce the greatest changes in a protein's properties are those in which: (a) a hydrophilic residue (e.g., Ser or Thr) is substituted for, or by, a hydrophobic residue (e.g. Leu, Ile, Phe or Val); (b) a cysteine or proline is substituted for, or by, any other residue, (c) a residue having an electropositive side chain (e.g., Arg, His or Lys) is substituted for, or by, an electronegative residue (e.g., Glu or Asp) or (d) a residue having a bulky side chain (e.g., Phe or Trp) is substituted for, or by, one having a smaller side chain (e.g., Ala, Ser) or no side chain (e.g., Gly). Variants may also comprise one or more amino acid deletions.

Substitutions preferably comprise those exemplified in the vacuole targeting motifs X₁X₂X₃PX₄ (SEQ ID NO:1) and/or (I/L)(R/K)LPS (SEQ ID NO:24).

It will be appreciated that isoleucine (I) and leucine (L) are both hydrophobic residues and that both arginine (R) and lysine (K) are both basic or positively charged residues, which comprise conservative substitutions. Thus vacuole targeting peptide motif is characterized by a general structure of “hydrophobic residue-basic residue-hydrophobic residue-proline (characterized by a bend structure)-hydrophilic residue”, which is susceptible to modification and variation while nevertheless retaining vacuolar targeting function.

Terms used herein to describe sequence relationships between respective nucleic acids and proteins include “comparison window”, “sequence identity”, “percentage of sequence identity” and “substantial identity”. Because respective nucleic acids/proteins may each comprise: (1) only one or more portions of a complete nucleic acid/protein sequence that are shared by the nucleic acids/proteins, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of typically at least 6 contiguous residues that is compared to a reference sequence. The comparison window may comprise additions or deletions (i.e., gaps) of about 20% or less as compared to the reference sequence (which does not comprise additions or deletions) for optimal alignment of the respective sequences. Optimal alignment of sequences for aligning a comparison window may be conducted by computerised implementations of algorithms (for example ECLUSTALW and BESTFIT provided by WebAngis GCG, 2D Angis, GCG and GeneDoc programs, incorporated herein by reference) or by inspection and the best alignment (i.e., resulting in the highest percentage homology over the comparison window) generated by any of the various methods selected.

The ECLUSTALW program is used to align multiple sequences. This program calculates a multiple alignment of nucleotide or amino acid sequences according to a method by Thompson, J. D., Higgins, D. G. and Gibson, T. J. (1994) and is part of an original ClustalW distribution, modified for inclusion in EGCG. The BESTFIT program aligns forward and reverse sequences and sequence repeats. This program makes an optimal alignment of a best segment of similarity between two sequences. Optimal alignments are determined by inserting gaps to maximize the number of matches using the local homology algorithm of Smith and Waterman. ECLUSTALW and BESTFIT alignment packages are offered in WebANGIS GCG (The Australian Genomic Information Centre, Building JO3, The University of Sydney, N.S.W 2006, Australia).

Reference also may be made to the BLAST family of programs as for example disclosed by Altschul et al., 1997, Nucl. Acids Res. 25 3389, including BLASTN and BLASTX databases located at NCBI (Altschul et al, 1990), which are incorporated herein by reference.

A detailed discussion of sequence analysis can be found in Chapter 19.3 of Ausubel et al, supra.

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, U) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA).

As generally used herein, a “homology” relates to a definable nucleotide or amino acid sequence relationship of an homologous protein or nucleic aid with a nucleic acid or protein of the invention as the case may be.

“Protein homologs” share at least 70%, preferably at least 80%, 85%, 90% and more preferably at least 95%, 96%, 97%, 98% or 99% sequence identity with the amino acid sequences of proteins of the invention as herein described.

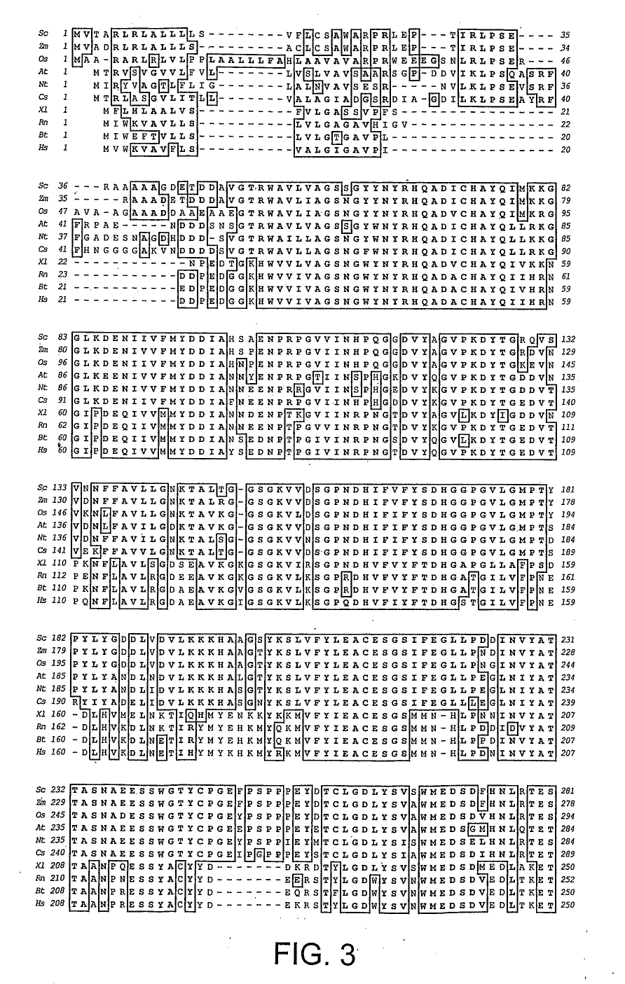

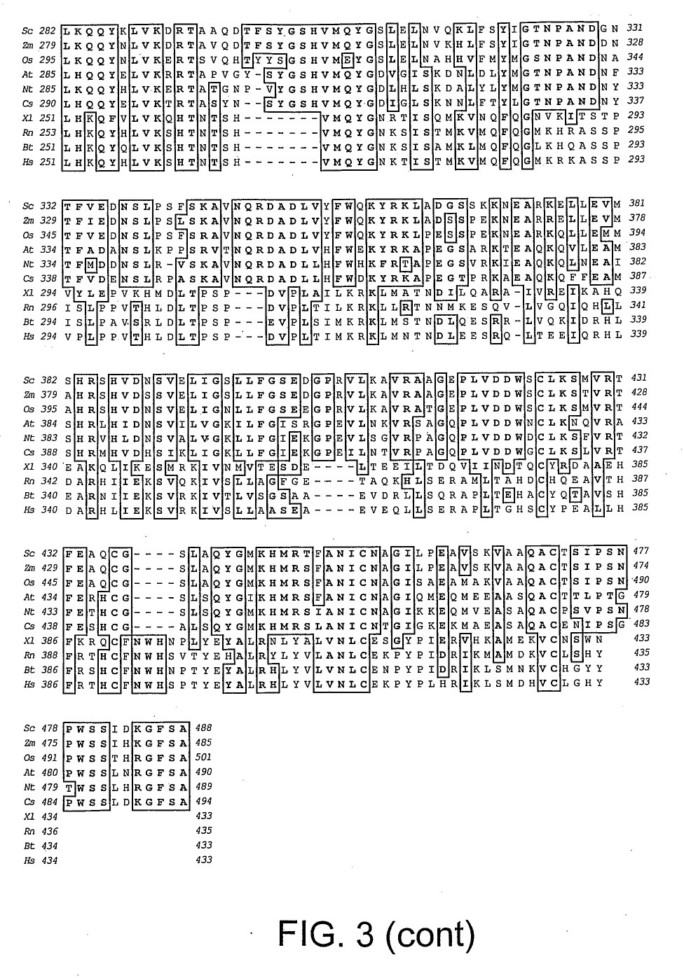

Preferably, a homolog comprises a percent homology between 70% and 99% and all values therebetween, for example the values recited above. Protein homologs include, for example proteins shown in FIG. 3.

Preferably, a homolog comprises a vacuole targeting peptide, more preferably further comprising a secretory signal peptide. Preferably, the vacuole targeting peptide comprises an amino acid motif X₁X₂X₃PX₄, and more preferably comprises an amino acid motif (I/L)(R/K)LPS (SEQ ID NO:24).

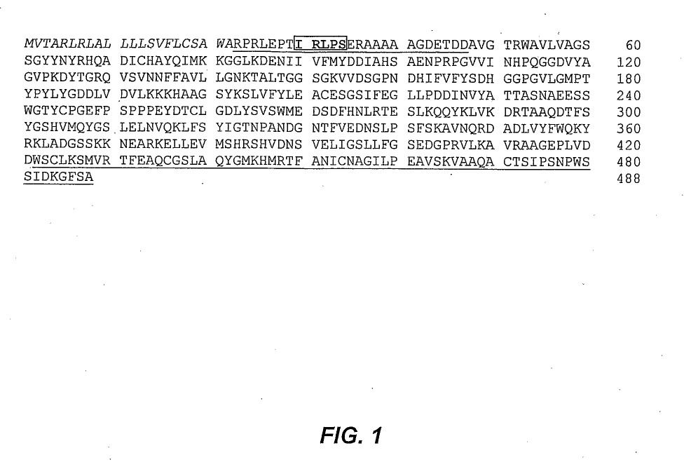

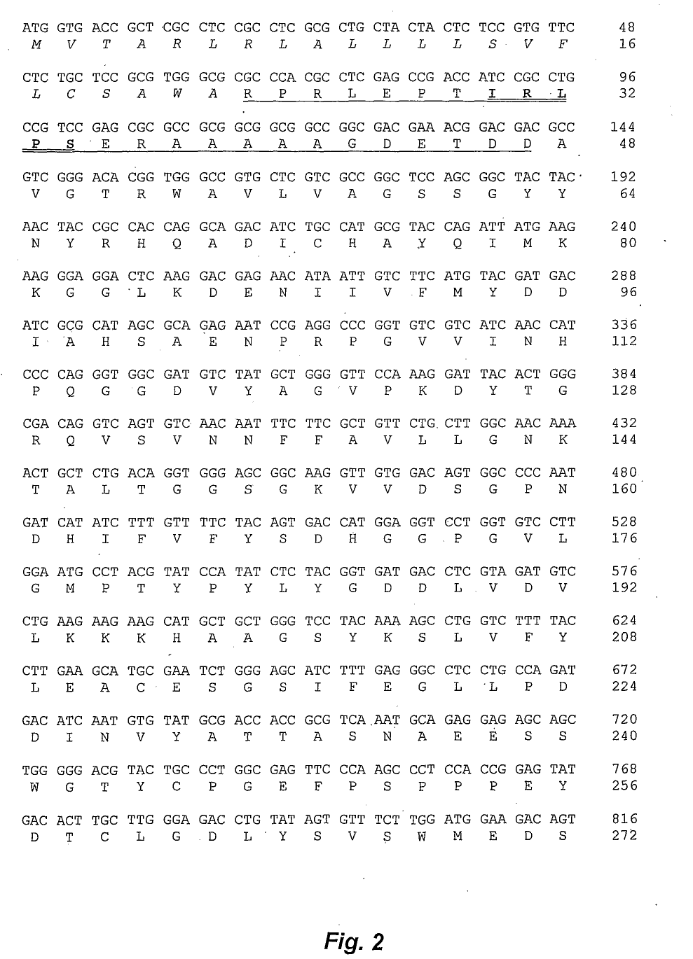

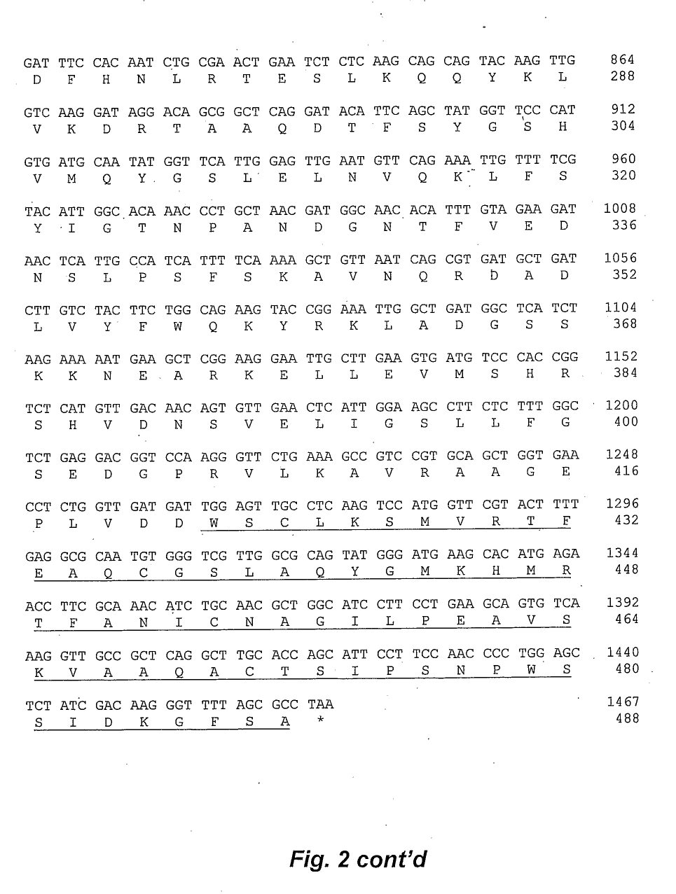

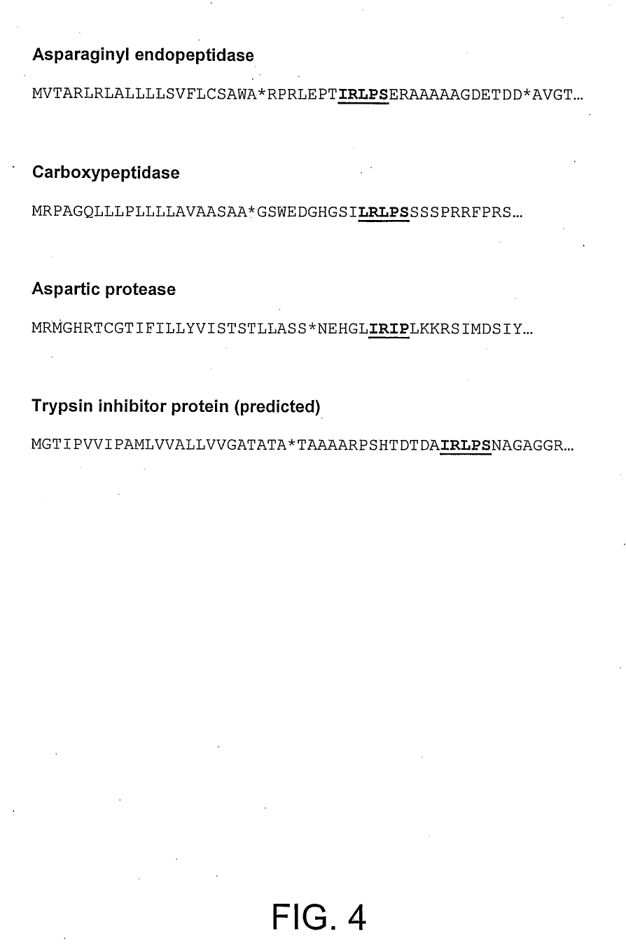

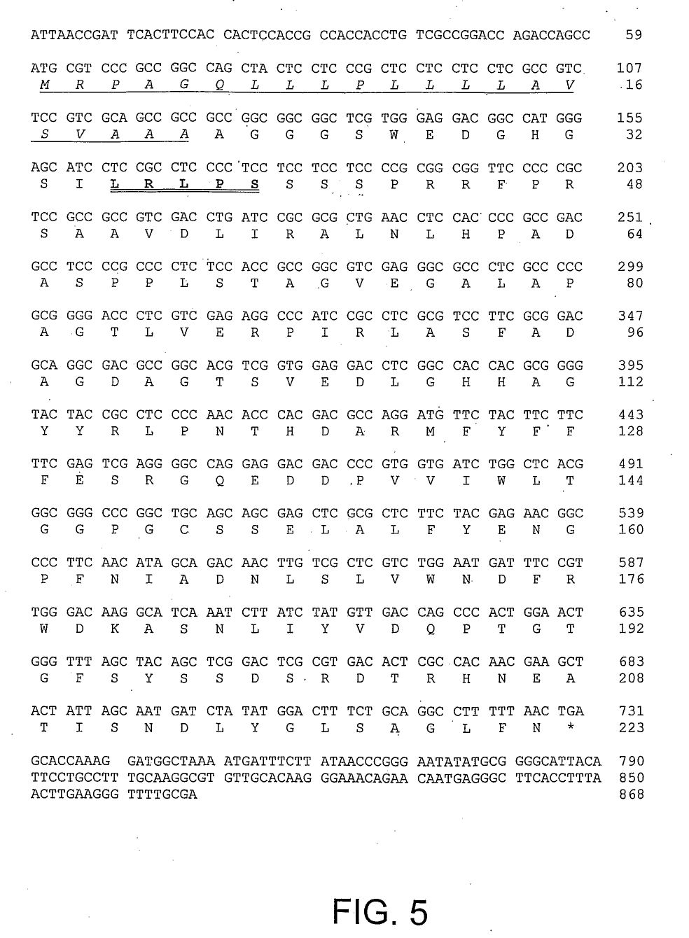

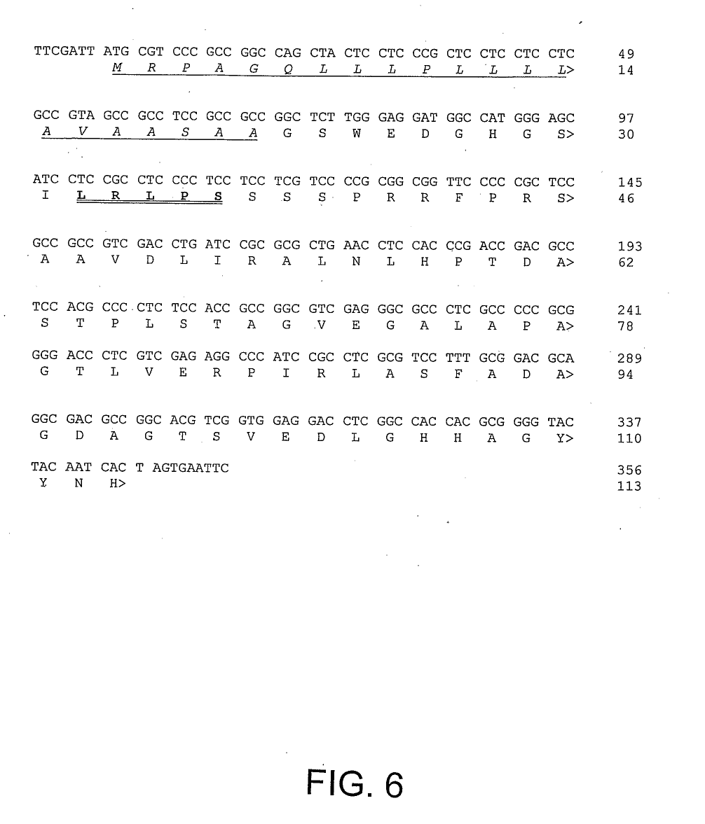

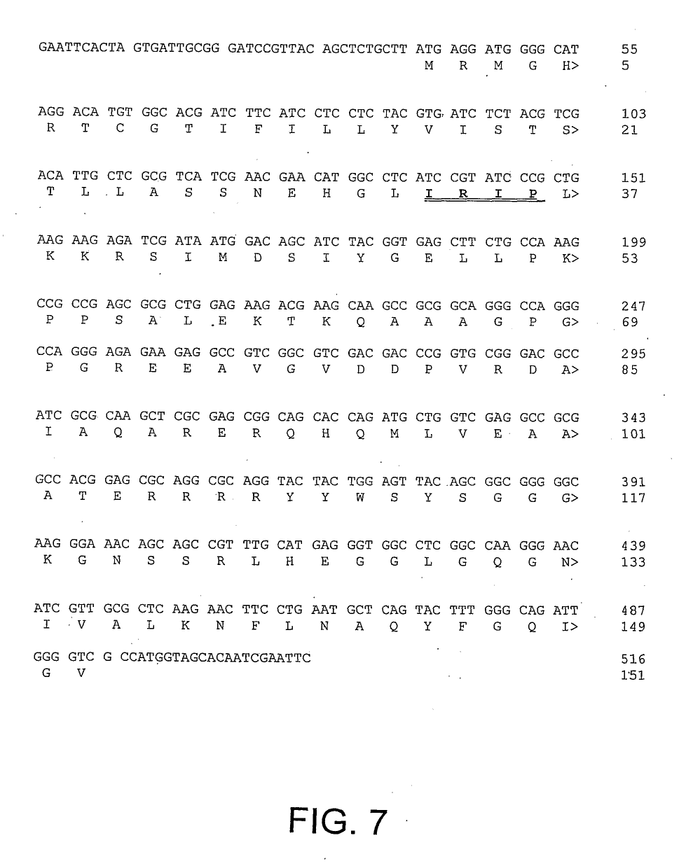

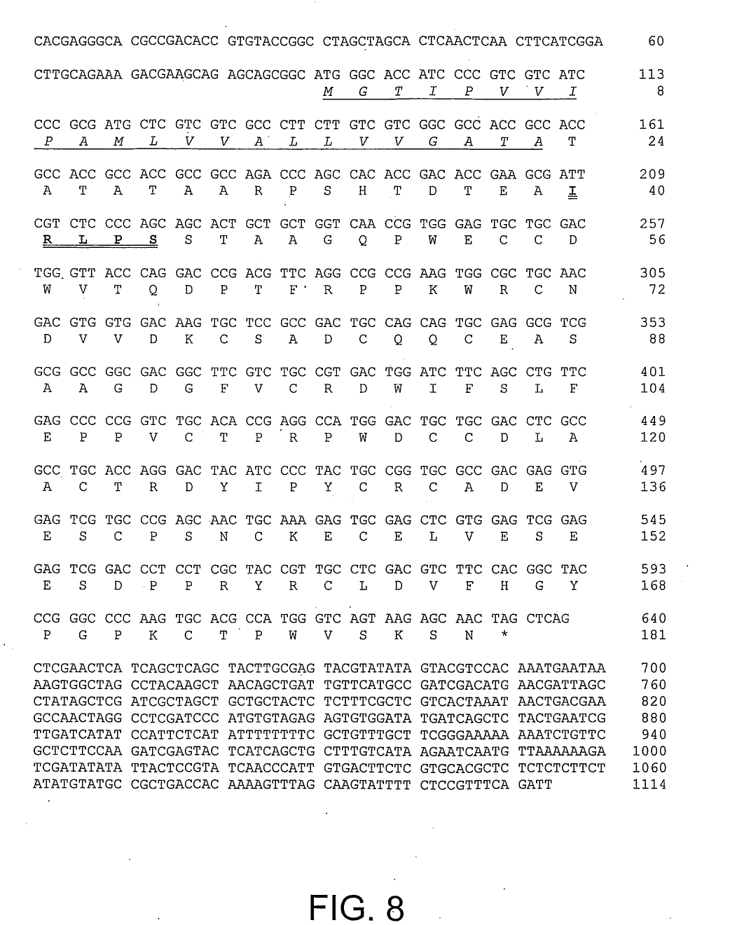

In a particular form, the invention contemplates isolated proteins, or fragments thereof, that are homologous to an N-terminal region of the endopeptidase protein shown in FIG. 1 or FIG. 2 (for example amino acids 1-87). or the N-terminal protease sequences shown in FIG. 4.

Included within the scope of homologs are “orthologs”, which are functionally-related proteins and their encoding nucleic acids, isolated from other organisms, for example as shown in FIG. 3. For example, orthologs obtainable from monocotyledonous plants such as sugarcane, wheat, rice, barley; dicotyledonous plants such as Arabidopsis, tobacco, sweet potato; animals such as frog, rat, mouse, cattle, human; bacteria; parasites and the like.

With regard to protein variants, these can be created by mutagenising a protein or by mutagenising an encoding nucleic acid, such as by random mutagenesis or site-directed mutagenesis. Examples of nucleic acid mutagenesis methods are provided in Chapter 9 of CURRENT PROTOCOLS IN MOLECULAR BIOLOGY, Ausubel et al., supra which is incorporated herein by reference.

It will be appreciated by the skilled person that site-directed mutagenesis is best performed where knowledge of the amino acid residues that contribute to biological activity is available. In many cases, this information is not available, or can only be inferred by molecular modeling approximations, for example.

In such cases, random mutagenesis is contemplated. Random mutagenesis methods include chemical modification of proteins by hydroxylamine (Ruan et al., 1997, Gene 188 35), incorporation of dNTP analogs into nucleic acids (Zaccolo et al., 1996, J. Mol. Biol. 255 589) and PCR-based random mutagenesis such as described in Stemmer, 1994, Proc. Natl. Acad. Sci. USA 91 10747 or Shafikhani et al., 1997, Biotechniques 23 304, each of which references is incorporated herein. It is also noted that PCR-based random mutagenesis kits are commercially available, such as the Diversify™ kit (Clontech).

As used herein, “derivative” proteins are proteins of the invention which have been altered, for example by conjugation or complexing with other chemical moieties or by post-translational modification techniques as would be understood in the art. Such derivatives include amino acid deletions and/or additions to proteins of the invention, or variants thereof.

“Additions” of amino acids may include fusion of the peptide or proteins or variants thereof with other peptides or proteins. Particular examples of such peptides include amino (N) and carboxyl (C) terminal amino acids added for use as “tags”. A tag preferably includes Green Fluorescent Protein (GFP), which is used as a marker for protein expression as described herein. Other tags include, for example, an N-terminal 6×-His tag for isolating an expressed fusion protein.

N-terminal and C-terminal tags include known amino acid sequences which bind a specific substrate, or bind known antibodies, preferably monoclonal antibodies. pRSET B vector (ProBond™; Invitrogen Corp.) is an example of a vector comprising an N-terminal 6×-His-tag which binds ProBond™ resin.

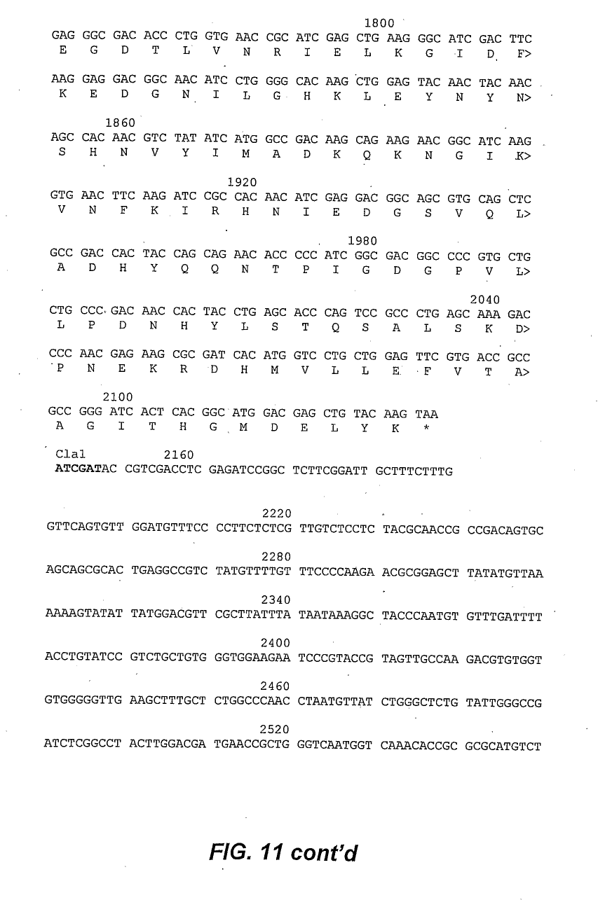

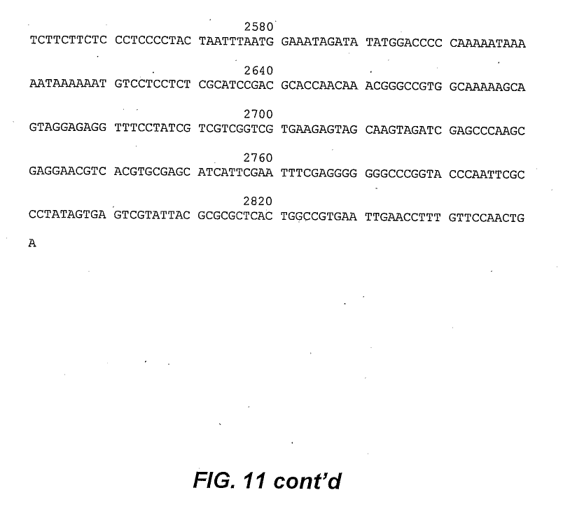

A “linker” amino acid or peptide comprises amino acid “additions”, but is not limited thereto. Although the linker amino acid or peptide in one form may comprise an amino acid addition not native or normally found contiguous with a peptide of interest, the linker in another form may comprise an N-terminal or C-terminal portion of the peptide of interest. For example, the linker may comprise an N-terminal fragment or portion of a peptide targeted for a vacuole, preferably the peptide comprises asparaginyl endopeptidase, carboxypeptidase, trypsin inhibitor protein or aspartic protease. An example of such a linker includes a peptide located between a vacuole targeting peptide and a heterologous d protein of interest. A linker may comprise, for example, amino acids 35-88 or amino acids 48-88 as shown in FIG. 1 or the linker sequences shown in FIGS. 11-15. A linker may comprise one or more amino acids, for example 1-100 amino acids and any value inclusive and therebetween, for example 1, 2, 3, 4, 5, 6, 7, 8, 9, 10, 15, 10, 30, 40, 50, 60, 70, 80, 90 or 100. The linker may be located at either or both N-terminal and/or C-terminal end of a heterologous protein, preferably, at the N-terminal end. More preferably, the linker is located between a vacuole targeting sequence and the heterologous protein. As such, an encoding nucleotide linker sequence may form part of a genetic construct.

Other derivatives contemplated by the invention include, modification to side chains, incorporation of unnatural amino acids and/or their derivatives during peptide or protein synthesis and the use of cross linkers and other methods which impose conformational constraints on the proteins, fragments and variants of the invention. Examples of side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2, 4, 6-trinitrobenzene sulphonic acid (TNBS).

The carboxyl group may be modified by carbodiimide activation via O-acylisourea formation followed by subsequent derivitization, by way of example, to a corresponding amide.

The guanidine group of arginine residues may be modified by formation of heterocyclic condensation products with reagents such as 2,3-butanedione, phenylglyoxal and glyoxal.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulphonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids.

Chimeric proteins of the invention may be prepared by any suitable procedure known to those of skill in the art.

For example, the protein may be prepared by a procedure including the steps of:
(i) preparing an expression construct which comprises a recombinant nucleic acid of the invention, operably linked to one or more regulatory nucleotide sequences, for example a T7 promoter;
(ii) transfecting or transforming the expression construct into a suitable host cell, for example E. coli; and
(iii) expressing the protein in said host cell.
Recombinant proteins may be conveniently expressed and purified by a person skilled in the art using commercially available kits, for example “ProBond™ Purification System” available from Invitrogen Corporation, Carlsbad, Calif., USA, herein incorporated by reference. Alternatively, standard molecular biology protocols may be used, as for example described in Sambrook, et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-1999), incorporated herein by reference, in particular Chapters 10 and 16; and CURRENT PROTOCOLS IN PROTEIN SCIENCE Eds. Coligan et al., (John Wiley & Sons, Inc. 1995-1999) which is incorporated by reference herein, in particular Chapters 1, 5, 6 and 7.

### Nucleic Acids

The invention provides an isolated nucleic acid that encodes a vacuole targeting sequence of the invention and/or a chimeric protein (“chimeric nucleic acid”) as hereinbefore described.

Such nucleic acids may be particularly useful for recombinant protein expression in plants for the purposes of vacuole targeting, or for production in vitro.

The term “nucleic acid” as used herein designates single or double stranded mRNA, RNA, cRNA and DNA, said DNA inclusive of cDNA and genomic DNA. A nucleic acid may be native or recombinant and may comprise one or more artificial nucleotides, e.g. nucleotides not normally found in nature. Nucleic acid encompasses modified purines (for example, inosine, methylinosine and methyladenosine) and modified pyrimidines (thiouridine and methylcytosine).

The term “isolated nucleic acid” as used herein refers to a nucleic acid subjected to in vitro manipulation into a form not normally found in nature. Isolated nucleic acid include both native and recombinant (non-native) nucleic acids. For example, a nucleic acid isolated from sugarcane, such as asparaginyl endopeptidase, carboxypeptidase, trypsin inhibitor protein or aspartic protease.

A “polynucleotide” is a nucleic acid having eighty (80) or more contiguous nucleotides, while an “oligonucleotide” has less than eighty (80) contiguous nucleotides.

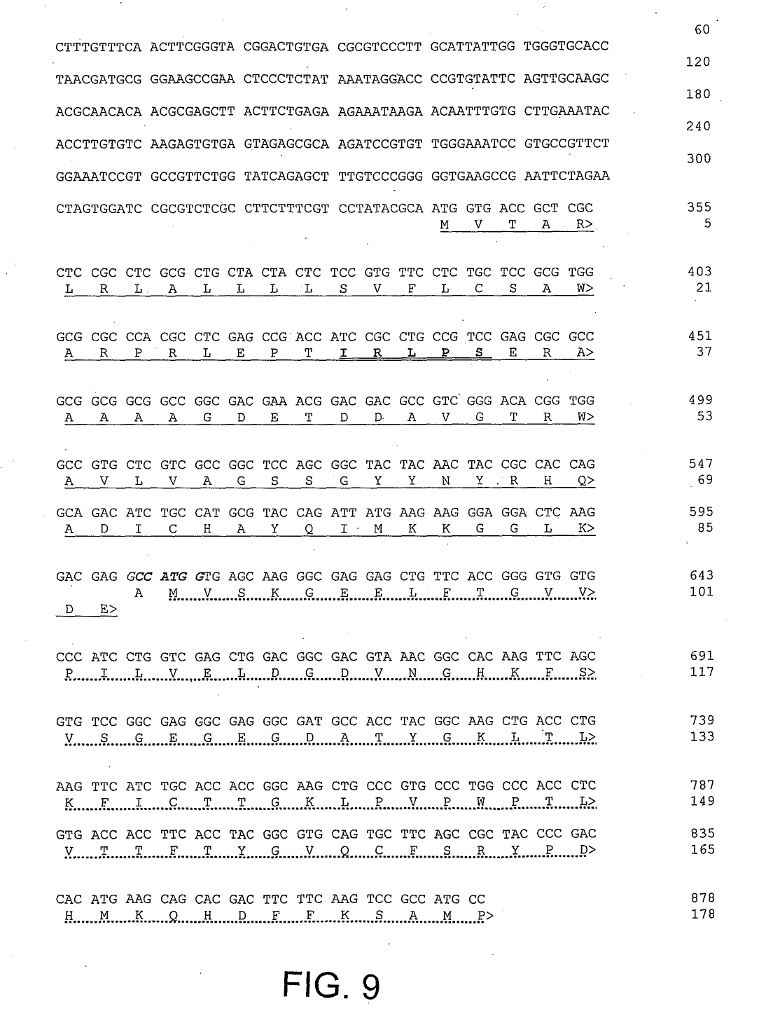

In one embodiment, a nucleic acid “fragment” comprises a nucleotide sequence that constitutes less than 100% of a nucleic acid of the invention, for example, less than or equal to: 99%, 98%, 95%, 90%, 85%, 80%, 75%, 70%, 65%, 60%, 55%, 50%, 45%, 40%, 35%, 30%, 25%, 20%, 15%, 10%, 8%, 6%, 4%, 2% or even 1%. It will be appreciated that a fragment comprises all integer values less than 100%, for example the percent value as set forth above and others. A fragment includes a polynucleotide, oligonucleotide, probe, primer and an amplification product, e.g. a PCR product. For example, a PCR fragment includes a fragment encoding an N-terminal portion of sugarcane asparaginyl endopeptidase, such as, a nucleic acid comprising a nucleotide sequence comprising 264 nucleotides encoding the secretory signal peptide, the putative vacuolar targeting motif and the first 40 amino acids of the mature asparaginyl endopeptidase protein as shown in FIG. 9.

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably comprising 20-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. For example, the following primers were used for PCR: 5′-CGTCTCGCCTTCTTTCGTCC (SEQ ID NO: 26), 5′-TGTAATGTAATGGAGTTCGGTGTGG (SEQ ID NO: 27), 5′-GCGGGATCCGCGTCTCGCCTTCTTTCGTCC (SEQ ID NO: 28) and 5′-GTGCTACCATGGCCTCGTCCTTGAGTCCTCC (SEQ ID NO: 29).

Primers may be used to amplify nucleic acids common to one or more species. A primer preferably comprises about 5 to 200 contiguous nucleotides, including all integer values inclusive and therebetween, for example, 5, 10, 20, 30, 40, 50, 75, 100, 125, 150, 150, 175 and 200.

As used herein, the term nucleic acid “variant” means a nucleic acid of the invention, the nucleotide sequence of which has been mutagenized or otherwise altered so as to encode substantially the same, or a modified protein. Such changes may be trivial, for example in cases where more convenient restriction endonuclease cleavage and/or recognition sites are introduced without substantially affecting biological activity of an encoded protein when compared to a non-variant form. Other nucleotide sequence alterations may be introduced so as to modify biological activity of an encoded protein. These alterations may include deletion or addition of one or more nucleotide bases, or involve non-conservative substitution of one base for another. Such alterations can have profound effects upon biological activity of an encoded protein, possibly increasing or decreasing biological activity. In this regard, mutagenesis may be performed in a random fashion or by site-directed mutagenesis in a more “rational” manner. Standard mutagenesis techniques are well known in the art, and examples are provided in Chapter 9 of CURRENT PROTOCOLS IN MOLECULAR BIOLOGY Eds Ausubel et al. (John Wiley & Sons NY, 1995), which is incorporated herein by reference.

A “genetic construct” preferably comprises a nucleic acid of the invention and one or more additional nucleotide sequences that facilitate manipulation, propagation and/or expression of the nucleic acid of the invention.

In a preferred embodiment, the genetic construct is an expression construct, wherein the isolated nucleic acid is operably linked or connected to one or more regulatory sequences in an expression vector.

In one preferred embodiment, the expression construct encodes the vacuolar targeting sequence set forth in SEQ ID NO:1, together with a cloning site (e.g. a polylinker), which facilitates “in frame” insertion of a heterologous nucleic acid to be expressed.

This embodiment is essentially an “off the shelf” construct that allows in frame insertion of any nucleic acid, having appropriate restriction sites, that encodes a heterologous protein of interest.

In another preferred embodiment, the expression construct comprises a “chimeric nucleic acid”. The chimeric nucleic acid preferably encodes the vacuolar targeting sequence set forth in SEQ ID NO:1 and a heterologous nucleic acid. The chimeric nucleic acid preferably further comprises a nucleic acid encoding a secretory signal peptide as described herein. Suitably, the expression construct facilitates targeting a heterologous protein of interest to a plant vacuole.

The heterologous protein of interest is preferably expressible so as to be isolated or purified from a plant vacuole.

Examples of expression constructs are gfp expression constructs as set forth in the Examples and SEQ ID NOS:53-55.

An “expression vector” may be either a self-replicating extra-chromosomal vector such as a plasmid, or a vector that integrates into a host genome. An example of an expression vector is pGEMT-easy (Promega), pCvGFPT, pRSET B (Invitrogen Corp.) and derivations thereof.

By “operably linked or connected” is meant that said one or more regulatory nucleotide sequence(s) is/are positioned relative to the recombinant nucleic acid of the invention to initiate, regulate or otherwise control transcription.

Regulatory nucleotide sequences will generally be appropriate for the host cell used for expression. Numerous types of appropriate expression vectors and suitable regulatory sequences are known in the art for a variety of host cells.

Typically, said one or more regulatory nucleotide sequences may include, promoter sequences, leader or signal sequences, ribosomal binding sites, transcriptional start and termination sequences, translational start and termination sequences, and enhancer or activator sequences.

Constitutive or inducible promoters as known in the art are contemplated by the invention. The promoters may be either naturally occurring promoters, or hybrid promoters that combine elements of more than one promoter. For example, the lac promoter is inducible by IPTG. An example of a suitable promoter is a banana streak virus promoter as described in Schenk et al, 2001 and a maize adh1 promoter (Chamberlain et al. 1994), both are incorporated herein by reference.

The expression vector may further comprise a selectable marker gene to allow the selection of transformed host cells. Selectable marker genes are well known in the art and will vary with the host cell used. For example, Neomycin Phosphotransferase II (nptII) gene that confers resistance to aminoglycosides, preferably, kanamycin, paromycin, neomycin and geneticin (G418) for selection of positively transformed host cells when grown in a medium comprising neomycin. The nptII gene may be under expression control of a promoter, for example a maize adh1 promoter (Chamberlain et al. 1994). Other selectable markers are well known in the art including: bar gene, ampicillin resistance gene and others.

The expression vector may also include a fusion partner (typically provided by the expression vector) so that the recombinant protein of the invention is expressed as a fusion protein with the fusion partner. An advantage of fusion partners is that they assist identification and/or purification of the fusion protein. Identification preferably includes visual inspection of fluorescence by GFP. Identification and/or purification may also include using a monoclonal antibody or substrate specific for the fusion partner, for example a 6×-His tag or GST. A fusion partner may also comprise a leader sequence for directing secretion of a recombinant protein, for example a secretory signal sequence as shown in FIG. 1 or an alpha-factor leader sequence. The fusion partner may also comprise a vacuole targeting sequence, for example, as shown in FIG. 1.

Well known examples of fusion partners include: GFP, hexahistidine (6×-HIS)-tag, N-Flag, Fc portion of human IgG, glutathione-S-transferase (GST) and maltose binding protein (MBP), which are particularly useful for isolation of the fusion protein by affinity chromatography. For the purposes of fusion protein purification by affinity chromatography, relevant matrices for affinity chromatography may include nickel-conjugated or cobalt-conjugated resins, fusion protein specific antibodies, glutathione-conjugated resins, and amylose-conjugated resins respectively. Some matrices are available in “kit” form, such as the ProBond™ Purification System (Invitrogene Corp.) which incorporates a 6X-His fusion vector and purification using ProBond™ resin.

In order to express the fusion protein, it is necessary to ligate a nucleic acid according to the invention into the expression vector so that the translational reading frames of the fusion partner and the nucleotide sequence of the invention coincide.

The fusion partners may also have protease cleavage sites, for example as shown in FIG. 4 by a star symbol. Other protease cleavage sites include enterokinase (available from Invitrogen Corp. as EnterokinaseMax™), Factor Xₐ or Thrombin, which allow the relevant protease to digest the fusion protein and thereby liberate the recombinant protein therefrom. The liberated protein can then be isolated from the fusion partner by subsequent chromatographic separation

Fusion partners may also include within their scope “epitope tags”, which are usually short peptide sequences for which a specific antibody is available.

As hereinbefore, proteins of the invention, such as chimeric proteins, may be produced by culturing a host cell transformed with an expression construct comprising a nucleic acid encoding the protein. The conditions appropriate for protein expression will vary with the choice of expression vector and the host cell. For example, a nucleotide sequence of the invention may be modified for successful or improved protein expression in a given host cell. Modifications include altering nucleotides depending on preferred codon usage of the host cell. Alternatively, or in addition, a nucleotide sequence of the invention may be modified to accommodate host specific splice sites or lack thereof. These modifications may be ascertained by one skilled in the art.

Host cells for expression may be prokaryotic or eukaryotic.

Useful prokaryotic host cells are bacteria.

A typical bacteria host cell is a strain of E coli.

Useful eukaryotic cells are yeast, plant cells, SF9 cells that may be used with a baculovirus expression system, and other mammalian cells. Plant cells preferably comprise callus cells.

The recombinant protein may be conveniently prepared by a person skilled in the art using standard protocols as for example described in Sambrook, et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-1999), incorporated herein by reference, in particular Chapters 10 and 16; and CURRENT PROTOCOLS IN PROTEIN SCIENCE Eds. Coligan et al., (John Wiley & Sons, Inc. 1995-1999) which is incorporated by reference herein, in particular Chapters 1, 5 and 6.

In one embodiment, nucleic acid homologs encode protein homologs of the invention, inclusive of variants, fragments and derivatives thereof.

In one embodiment, nucleic acid variants are nucleic acids having one or more codon sequences altered by taking advantage of codon sequence redundancy. For this embodiment, the homologous nucleotide sequence may be different from a wild-type sequence, but still encode a same protein or peptide.

A particular example of this embodiment is optimization of a nucleic acid sequence according to codon usage as is well known in the art. This can effectively “tailor” a nucleic acid for optimal expression in a particular organism, or cells thereof, where preferential codon usage has been established. For example, a nucleotide sequence may be optimized for a monocotyledon such as sugarcane, maize, wheat, barley or a dicotyledon such as Arabidopsis or tobacco.

In one embodiment, nucleic acid homologs share at least 60%, preferably at least 70%, more preferably at least 80%, 85%, and even more preferably at least 90%, 91%, 92%, 93%, 94%, 95%, 96%, 97%, 98% or 99% sequence identity with the nucleic acids of the invention. Preferably, the nucleic acid homolog comprises a percent identity between 60% and less than 100%, inclusive of all values therebetween, for example as shown above.

In another embodiment, nucleic acid homologs hybridize to nucleic acids of the invention under at least low stringency conditions, preferably under at least medium stringency conditions and more preferably under high stringency conditions.

“Hybridise and Hybridisation” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing.

Modified purines (for example, inosine, methylinosine and methyladenosine) and modified pyrimidines (thiouridine and methylcytosine) may also engage in base pairing.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“Stringent conditions” designates those conditions under which only nucleic acid having a high frequency of complementary bases will hybridize.

Reference herein to high stringency conditions include and encompass:—
(i) from at least about 31% v/v to at least about 50% v/v formamide and from at least about 0.01 M to at least about 0.15 M salt for hybridisation at 42° C., and at least about 0.01 M to at least about 0.15 M salt for washing at 42° C.;
(ii) 1% BSA, 1 mM EDTA, 0.5 M NaHPO₄ (pH 7.2), 7% SDS for hybridization at 65° C., and (a) 0.1×SSC, 0.1% SDS; or (b) 0.5% BSA, 1 mM EDTA, 40 mM NaHPO₄ (pH 7.2), 1% SDS for washing at a temperature in excess of 65° C. for about one hour; and
(iii) 0.2×SSC, 0.1% SDS for washing at or above 68° C. for about 20 minutes.

In general, the Tₘ of a duplex DNA decreases by about 1° C. with every increase of 1% in the number of mismatched bases.

Notwithstanding the above, stringent conditions are well known in the art, such as described in Chapters 2.9 and 2.10 of Ausubel et al., supra, which are herein incorporated by reference. A skilled addressee will also recognize that various factors can be manipulated to optimize the specificity of the hybridization. Optimization of the stringency of the final washes can serve to ensure a high degree of hybridization.

Typically, complementary nucleotide sequences are identified by blotting techniques that include a step whereby nucleotides are immobilized on a matrix (preferably a synthetic membrane such as nitrocellulose), a hybridization step, and a detection step.

Methods for detecting labeled nucleic acids hybridised to an immobilised nucleic acid are well known to practitioners in the art. Such methods include autoradiography, chemiluminescent, fluorescent and colourimetric detection.

Nucleic acid homologs of the invention may be prepared according to the following procedure:
(i) obtaining a nucleic acid extract from a suitable host, for example a plant species;
(ii) creating primers which are optionally degenerate, wherein each comprises a portion of a nucleotide sequence of the invention; and
(iii) using said primers to amplify, via nucleic acid amplification techniques, one or more amplification products from said nucleic acid extract.

As used herein, an “amplification product” refers to a nucleic acid product generated by nucleic acid amplification techniques.

Suitable nucleic acid amplification techniques are well known to the skilled addressee, and include PCR as for example described in Chapter 15 of Ausubel et al. supra, which is incorporated herein by reference; strand displacement amplification (SDA) as for example described in U.S. Pat. No. 5,422,252 which is incorporated herein by reference; rolling circle replication (RCR) as for example described in Liu et al., 1996, J. Am. Chem. Soc. 118 1587 and International application WO 92/01813; and Lizardi and Caplan, International Application WO 97/19193, which are incorporated herein by reference; nucleic acid sequence-based amplification (NASBA) as for example described by Sooknanan et al., 1994, Biotechniques 17 1077, which is incorporated herein by reference; ligase chain reaction (LCR) as for example described in International Application WO89/09385 which is incorporated herein by reference; and Q-β replicase amplification as for example described by Tyagi et al., 1996, Proc. Natl. Acad. Sci. USA 93 5395 which is incorporated herein by reference.

Preferably, amplification is by PCR using primers disclosed herein.

A microarray uses hybridization-based technology that, for example, may allow detection and/or isolation of a nucleic acid by way of hybridization of complementary nucleic acids. A microarray provides a method of high throughput screening for a nucleic acid in a sample that may be tested against several nucleic acids attached to a surface of a matrix or chip. In this regard, a skilled person is referred to Chapter 22 of CURRENT PROTOCOLS IN MOLECULAR BIOLOGY (Eds. Ausubel et al. John Wiley & Sons NY, 2000). A microarray may be used to isolate homologous nucleic acids of the present invention in same of different species.

### Genetically-Modified Plants

Other aspects of the present invention relate to genetically-modified or “transgenic” plants and a method of producing genetically modified plants.

In one embodiment, the method of producing a transgenic plant includes the steps of:—
(i) transforming a plant cell or tissue with an expression construct which comprises an isolated nucleic acid encoding a chimeric protein of the invention; and
(ii) selectively propagating a transgenic plant from the plant cell or tissue transformed in step (i).

Suitably, the plant cell or tissue used at step (i) may be leaf disk, callus, meristem, root, leaf spindle or whorl, leaf blade, stem, shoot, petiole, axillary bud, shoot apex, internode, flower stalk or inflorescence tissue.

Preferably, the tissue is callus.

The plant cell or tissue may be obtained from any plant species including monocotyledon, dicotyledon, ferns and gymnosperms such as conifers, without being limited thereto.

Preferably, the plant is a monocotyledon or dicotyledon.

Preferably, the monocotyledon is a species of sugarcane.

More preferably, the monocotyledon is a species of a sugarcane complex selected from the group consisting of the genera Saccharum, Erianthus, Miscanthus, Sclerostachya, Narenga and hybrids of these species.

Even more preferably, the sugarcane is Saccharum hybrid variety Q117.

Preferably, the dicotyledon is Arabidopsis or tobacco.

More preferably, the tobacco is Nicotianna tabacum.

For the purposes of producing a genetically-modified plant, the expressed nucleic acid encodes a chimeric protein comprising an amino acid sequence of a heterologous protein.

Preferably, the heterologous protein may be any protein of interest including a protein selected from the group consisting of: a sucrose modifying enzyme, a hexose modifying enzyme, a protein capable of use as an industrial enzyme, a protein capable of use as a pharmaceutical composition and/or diagnostic reagent, a protein capable of use in crop protection, a protein characterized by culinary or industrial properties and a vacuolar metabolite modifying enzyme as described herein.

For the purposes of introducing a genetic construct of the invention to a plant cell or tissue, a plant “transformation” method may be suitable employed.

Persons skilled in the art will be aware that a variety of transformation methods are applicable to the method of the invention, such as Agrobacterium-mediated (Gartland & Davey, 1995, Agrobacterium Protocols (Human Press Inc. NJ USA); U.S. Pat. No. 6,037,522; WO99/36637), microprojectile bombardment (Franks & Birch, 1991, Aust. J. Plant. Physiol., 18 471; Bower et al., 1996, Molecular Breeding, 2 239; Nutt et al., 1999, Proc. Aust. Soc. SugarCane Technol. 21 171), liposome-mediated (Ahokas et al., 1987, Heriditas 106 129), laser-mediated (Guo et al., 1995, Physiologia Plantarum 93 19), silicon carbide or tungsten whiskers (U.S. Pat. No. 5,302,523; Kaeppler et al., 1992, Theor. Appl. Genet. 84 560), virus-mediated (Brisson et al., 1987, Nature 310 511), polyethylene-glycol-mediated (Paszkowski et al., 1984, EMBO J. 3 2717) as well as transformation by microinjection (Neuhaus et al., 1987, Theor. Appl. Genet. 75 30) and electroporation of protoplasts (Fromm et al., 1986, Nature 319 791), all of which references are incorporated herein.

With particular regard to monocotyledons, sugarcane callus transformation is shown in the Examples herein. Other monocotyledons may likewise be transformed, for example, cereal grains such as maize, wheat, rice, barley, sorghum, rye, oats and the like. Dicotyledons, for example, tobacco, Arabidopsis, potato and the like, may likewise be transformed as discussed in (Horsch et al., 1985, Science 227 1229; Fry et al., 1987, Plant Cell Rep. 6 321), which are incorporated herein by reference. Although microprojectile bombardment is preferable for monocotyledons, microprojection and Agrobacterium transformation are also useful for transforming dicotyledons.

Preferably, microprojectile bombardment is used at transformation step (i). Generally, this is the preferred method for monocot transformation, as some monocot species have proven refractory to transformation by methods such as Agrobacterium-mediated transformation. However, recent success has been achieved with certain monocots (see for example U.S. Pat. No. 6,037,522 in relation to cereals and WO99/36637 in relation to pineapples), incorporated herein by reference, so that Agrobacterium-mediated transformation of monocots is contemplated by the present invention.

Preferably, selective propagation at step (ii) is performed in a selection medium which includes geneticin as selection agent.

In a preferred embodiment, a separate selection construct is included at step (i), which comprises a selection marker nucleic acid in the form of an nptII gene. More preferably, the selection construct comprises a plasmid pEMU, Which encodes the nptII gene.

In another embodiment, the expression construct further comprises a selection marker nucleic acid in the form of an nptII gene.

However, it will be appreciated that as discussed hereinbefore, there are a number of different selection agents useful according to the invention, the choice of selection agent being determined by the selection marker nucleic acid used in the expression construct or provided by a separate selection construct.

A transgenic plant comprises a transgenic plant cell, tissue, fruit or other plant part, which preferably expresses an isolated nucleic acid or genetic construct as described herein in relation to the invention.

### Vacuole Targeting and Isolating an Expressed Heterologous Protein

The invention in a preferred form relates to targeting an expressed heterologous protein of interest to a vacuole of a plant by fusing the expressed protein with the vacuole targeting sequence (SEQ ID NO:1) of the present invention.

The expressed, chimeric protein (i.e in recombinant form) preferably comprises a heterologous protein to be isolated, purified or otherwise obtained from a plant vacuole. The heterologous protein may be any protein, including a protein normally expressed in the transgenic plant or a transgene that is not normally expressed in the transgenic plant. If the expressed heterologous protein is normally expressed in the transgenic plant, the amount of the expressed protein is preferably greater than normal wild-type expression. Preferably, the amount of expressed protein is increased by increased translation and/or transcription, for example via a highly active promoter of an expression construct encoding the expressed, heterologous protein. Alternatively, or in addition, the expressed heterologous protein may not normally be targeted to a vacuole and fusion of the vacuole targeting peptide directs the expressed heterologous protein to the vacuole as described herein.

In a preferred form of the invention, a transgenic plant comprises a genetic construct encoding a chimeric protein comprising the vacuole targeting peptide as described herein (SEQ ID NO:1) and an additional expressed protein of interest. More preferably, the transgenic plant is characterized by substantially normal growth and development when compared with a wild-type non-transformed plant. In one preferred form, carbon flow is directed away from sucrose accumulation to produce an alternative product.

Examples of proteins of interest include, (1) sucrose modifying enzymes such as sucrose isomerase (preferably capable of producing isomaltulose), fructosyl transferases (preferably capable of producing fructans), invertase (preferably capable of producing hexoses), amylosucrase, dextransucrase and glucan sucrase (preferably capable of producing glucose polymers); (2) enzymes that preferably directly modify hexoses including for example polyol dehydrogenase, dextran synthases and other transferases (3) proteins for use as industrial enzymes including lipases, cellulase, pectinase, hemicellulase, peroxidases, amylase, dextranase, protease, polysaccharases, lytic enzymes, and others; (4) proteins for pharmaceutical/clinical/pathological and diagnostic purposes including antigens, antibodies, cytotoxic agents, anticancer proteins and vaccines; (5) proteins for crop protection including antifungal proteins (such as plant defensins), antibacterial proteins (such as thionins), anti-insect proteins (such as Bt, protease inhibitors, avidin) and anti-nematode proteins (such as collagenase); (6) proteins with particular culinary or industrial qualities including coagulants, gelling proteins, sweet proteins, sour proteins, adhesive proteins; and (7) enzymes that modify other vacuolar metabolites such as phenolics, tannins, flavonoids; and other secondary metabolites.

The transformed plant is preferably a monocotyledon or dicotyledon plant. Preferably, the monocotyledon plant is sugarcane, maize, wheat, barley, sorghum, rye, oats or rice.

In one form, the monocotyledon is preferably a cereal grain.

It will be appreciated by a skilled person that perturbation of sucrose metabolism in transgenic plants can be detrimental to normal plant function, e.g. normal growth and development. Accordingly, isolating expression of a recombinant peptide in a vacuole of a transgenic plant minimizes or avoids disruption of normal plant growth.

### Detection of Transgene Expression

The genetically-modified or “transgenic” status of plants of the invention may be ascertained by measuring, detecting or identifying transgenic expression of an expressed protein or an isolated nucleic acid encoding same.

For example, the isolated nucleic acid may be encoded be a transcribed nucleic acid (e.g. mRNA). This can be performed using the aforementioned methods applicable to detecting and measuring GFP activity and detection of a selectable marker. GFP fluorescence is preferably monitored in callus cultures using a Leica MZ6 dissecting microscope with a GFP PLUS fluorescence module (Leica AG, Heerbrugg, Switzerland). Cells are preferably examined with a Zeiss Axioskop epi-fluorescence microscope (Carl Zeiss Australia, North Ryde, NSW, 2113) fitted with a blue fluorescence excitation filter for detection of GFP or a UV excitation filter for detection of other dyes.

In one embodiment, transgene expression can be detected by antibodies specific for the encoded protein:
(i) in an ELISA such as described in Chapter 11.2 of CURRENT PROTOCOLS IN MOLECULAR BIOLOGY Eds. Ausubel et al. (John Wiley & Sons Inc. NY, 1995) which is herein incorporated by reference; or
(ii) by Western blotting and/or immunoprecipitation such as described in Chapter 12 of CURRENT PROTOCOLS IN PROTEIN SCIENCE Eds. Coligan et al. (John Wiley & Sons Inc. NY, 1997), which is herein incorporated by reference.

Protein-based techniques such as mentioned above may also be found in Chapter 4.2 of PLANT MOLECULAR BIOLOGY: A Laboratory Manual, supra, which is herein incorporated by reference.

Particularly advantageous protein assays preferably detect nptII-expressing transgenic plants.

The aforementioned protein-based detection methods may take advantage of “fusion partners” such as GFP, glutathione-S-transferase (GST), Fc portion of human IgG, maltose binding protein (MBP) and hexahistidine (HIS₆). For the purposes of fusion protein purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system.

In another form, a transgene may be detected by measuring a product produced by a reaction involving a protein expressed by the transgene. For example, in a preferred form the transgene encodes an enzyme and a product resulting from biolocial activity of the encoded enzyme is measured. In a more preferred form, the transgene encodes a fructosyl transferase protein and the product comprises fructan. Preferably, the fructosyl transferase protein comprises bacterial fructosyl transferase protein. Preferably, product is measured by chromatography. Preferably, the chromatography comprises high pressure liquid chromatography, gas chromatography and thin layer chromatography. More preferably, the fructan is measured by thin layer chromatography.

It will also be appreciated that transgenic plants of the invention may be screened for the presence of mRNA corresponding to a transcribable nucleic acid and/or a selection marker nucleic acid. This may be performed by RT-PCR and/or Northern hybridization. Southern hybridization and/or PCR may be employed to detect DNA (the vacuole targeting sequence, transcribable nucleic acid and/or selectable marker) in the transgenic plant genome.

As mentioned previously, PCR is a technique well known in the art and the aforementioned incorporated references provide exemplary PCR methods applicable to the present invention.

Particularly advantageous PCR assays preferably detect nptII-expressing transgenic plants.

For examples of RNA isolation and Northern hybridization methods, the skilled person is referred to Chapter 3 of PLANT MOLECULAR BIOLOGY: A Laboratory Manual, supra, which is herein incorporated by reference. Southern hybridization is described, for example, in Chapter 1 of PLANT MOLECULAR BIOLOGY: A Laboratory Manual, supra, which is herein incorporated by reference.

In order that the invention may be readily understood and put into practical effect, particular preferred embodiments will now be described by way of the following non-limiting examples.

### EXAMPLE 1

### Materials and Methods

Source of cDNA Clones

As described in Casu et al. (2003), incorporated herein by reference, a cDNA library was constructed from mRNA samples isolated from maturing stem (internodes 6-11) from 12-month old plants of sugarcane variety Q117. Random clones were subjected to single pass sequencing, the trace files were edited and the extracted sequences then analysed by homology searching of the non-redundant DNA, EST (both BLASTN) and protein (BLASTX) databases (Altschul et al., 1990) located at NCBI, incorporated herein by reference. All ESTs were extensively annotated for possible function and/or role by a combination of automated filtering and manual inspection, and were also clustered into contigs with gcphrap (http://www.phrap.org/—deviation from default settings: gap penalty 15, shatter_greedy, a bandwidth of 30 and a minimum score of 100). Multiple sequence alignment was done with the CLUSTALW algorithm as implemented in MacVector 7.0 (Accelrys, San Diego, Calif.).

### Recovery of Full-Length Clone

A contig encoding a hypothetical full-length sequence was constructed from sequences in public databases. Two PCR primers (sequences 5′-CGTCTCGCCTTCTTTCGTCC-3′ (SEQ ID NO: 26) and 5′-TGTAATGTAATGGAGTTCGGTGTGG-3′ (SEQ ID NO: 27) were used to generate a full-length clone from sugarcane stem cDNA produced with Superscript II (Invitrogen Australia Pty Ltd, Mt. Waverley 3149, Australia). The fragment was cloned into pGEMT-easy (Promega) according to the manufacturer's instructions.

### Growth and Transformation of Sugarcane Callus

Callus was transformed by microprojectile bombardment with plasmid DNA following the method of Bower et al. (1996), incorporated herein by reference. Tissue was co-bombarded with plasmid pEMU which encodes the nptII gene conferring antibiotic resistance, under the control of the maize adh1 promoter (Chamberlain et al. 1994), incorporated herein by reference.

### Microscopy and Cytochemistry

GFP fluorescence was monitored in callus cultures using a Leica MZ6 dissecting microscope with the GFP PLUS fluorescence module (Leica AG, Heerbrugg, Switzerland). Cells were examined with a Zeiss Axioskop epi-fluorescence microscope (Carl Zeiss Australia, North Ryde, NSW, 2113) fitted with a blue fluorescence excitation filter for detection of GFP or a UV excitation filter for detection of other dyes. Photographs were taken with an Olympus DP-70 digital camera.

The following stains were purchased from Molecular Probes (Invitrogen, Mt. Waverley, Vic. 3149, Australia) and used according to the manufacturer's instructions: the vacuolar lumen marker, CellTracker Blue CMAC (7-amino-4-chloromethyl-coumarin) and the yeast vacuole membrane marker, MDY-64.

### Reporter Gene Constructs

GFP reporter constructs designed to (1) secrete GFP into the apoplastic space and (2) retain GFP in the endoplasmic reticulum were prepared using plasmid pCvgfpt as a template for PCR reactions.

An initial PCR reaction was performed using the primers GFP-Fsp and GFP Rterm which consist of the sequences 5′ CTC TGC TCC GCT TGG GCT CGT GGA TCC GGA GCT AGC MG GGC GAG GAG CTG TTC 3′ (SEQ ID NO:45) and 5′ GTC GTA GCA GAT ACC ACT CT 3′ (SEQ ID NO:46) respectively.

The forward primer consists of a 27 nt region designed to anneal to the GFP sequence (bolded) and an additional 27 nt region corresponding to the last 6 amino acids of the endopeptidase signal peptide plus the adjacent amino acid thus preserving the native signal peptide cleavage site. In addition a small linker representing a BamH1 site (italics) was incorporated adjacent the GFP sequence to enable further cloning as required.

Nested primer pairs were then utilised for a second round of PCR. Primer SigF consisting of the sequence 5′ACT AGT ATG GTG ACC GCT CGC CTC CGC CTC GCG CTG CTA CTA CTC TCC GTG TTC CTC TGC TCC GCG TGG GCG CGC 3′ (SEQ ID NO:47) represents the native endopeptidase signal peptide. A Spe1 restriction enzyme site was incorporated (italics) at the 5′ end to allow cloning.

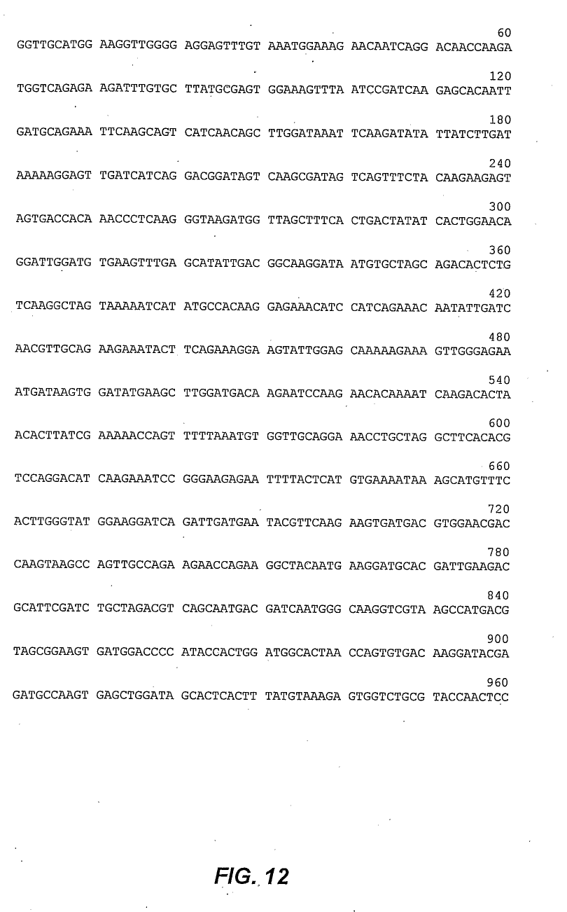

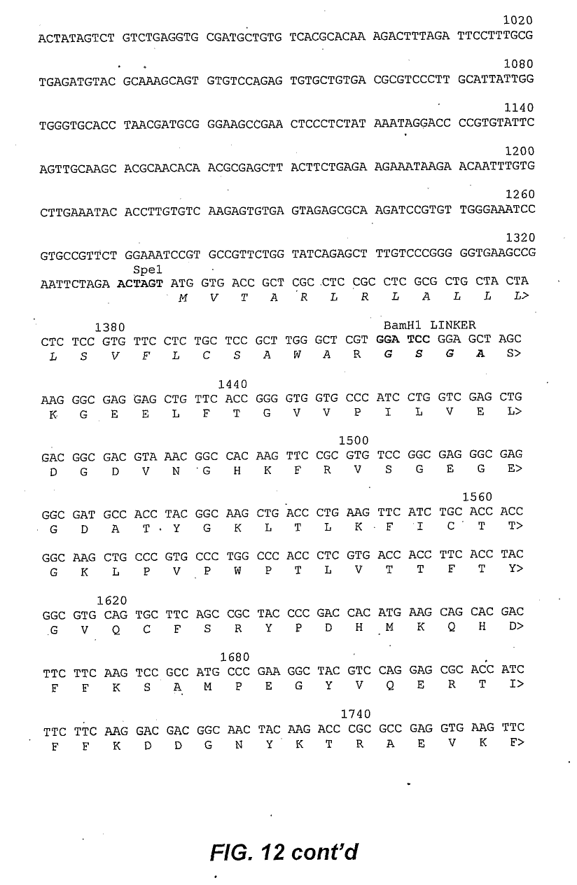

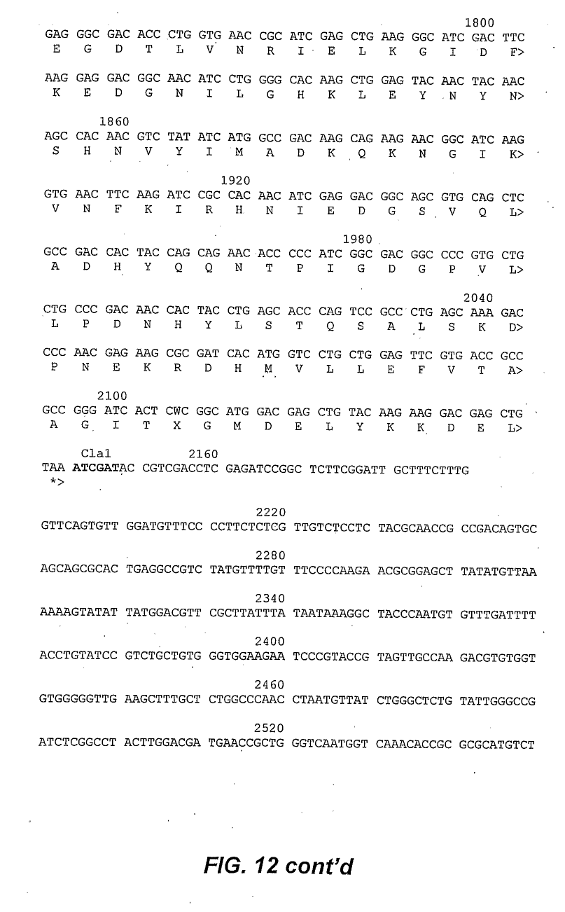

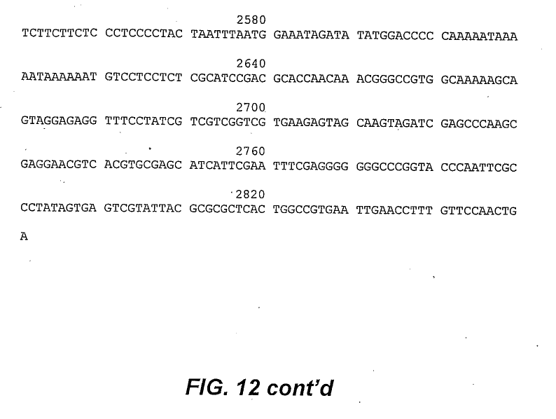

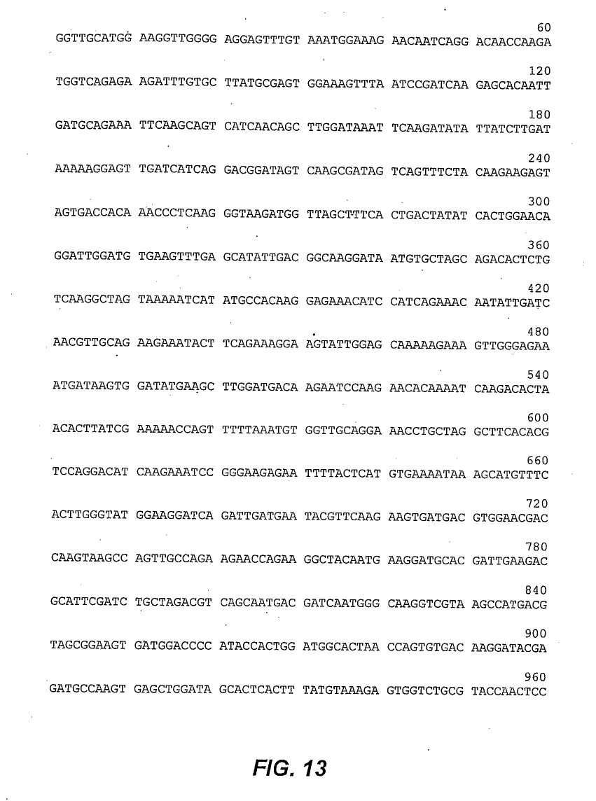

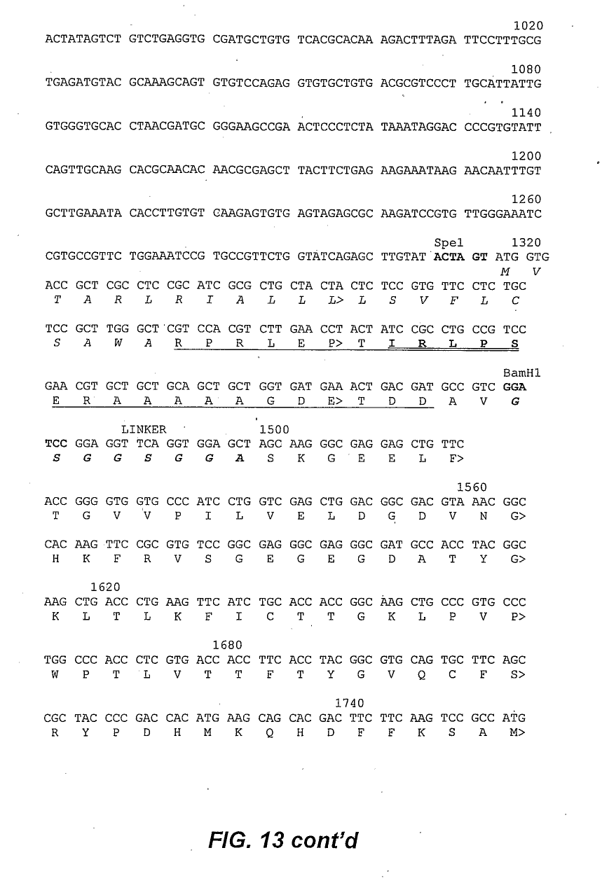

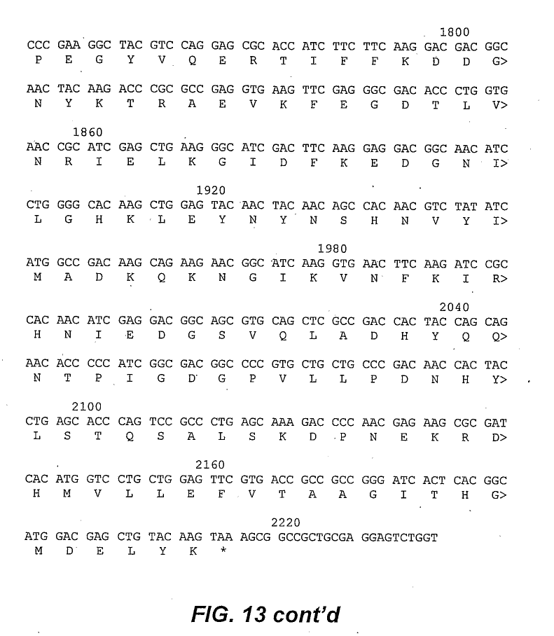

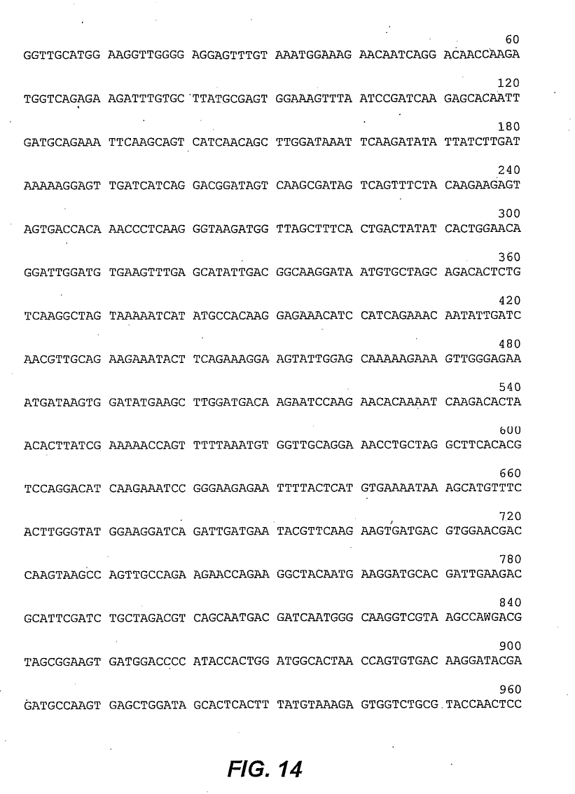

Reverse primers used included GFPRevCla1 and GFPRevKDELCla1, containing the sequences 5′GCG ATC GAT TTA CTT GTA CAG CTC GTC CA 3′ (SEQ ID NO:48) and 5′ GCG ATC GAT TTA CAG CTC GTC CTT CTT GTA CAG CTC GTC CAT GCC 3′ (SEQ ID NO:49) respectively. A Cla1 restriction site was incorporated (italics) to allow sub cloning. Shown in bold is the sequence corresponding to the KDEL motif used for ER retention of GFP. Digestion of PCR products with Spe1 and Cla1 and subsequent ligation back into the likewise digested pCvgfpt resulted in the completion of a secreted gfp construct (pCvsgfp; SEQ ID NO:53 & FIG. 11) and an ER retained GFP (pCvsgfpKDEL; SEQ ID NO:54 & FIG. 12).

To test the vacuolar targeting ability of the N terminus of the sugarcane endopeptidase gene primers were designed to amplify a 261 bp fragment consisting of both the signal peptide and full-predicted N terminal propeptide together with an additional 40 amino acids of the mature protein. Primers utilised included EndoForBam and EndoRevNco1 corresponding to 5′-GCG GGA TCC GCG TCT CGC CTT CTT TCG TCC-3′ (SEQ ID NO: 28) and 5′-GTG CTA CCA TGG CCT CGT CCT TGA GTC CTC C-3′ (SEQ ID NO: 29) respectively. This fragment was cloned in frame at the 5′ end of the S65T-GFP reporter gene in plasmid pCvGFPT to produce pCvEndoNTPP-gfp which is under the control of the banana streak virus promoter (Schenk et al. 2001).

To further analyse the putative vacuolar-targeting motif within the endopeptidase NTPP 3 more GFP reporter constructs were designed and synthesised. The coding preference of the endopeptidase NTPP was altered to decrease the GC content as initial cloning attempts resulted in nucleotide deletions. In all cases overlapping oligonucleotides were synthesised, annealed and extended using PCR. For plasmid pCvEndoExp1-gfp (SEQ ID NO: 55 & FIG. 13) two PCR reactions were needed. Primers Exp1For#1 and Exp1Rev#2 representing the sequences 5′ TTC CTC TGC TCC GCG TGG GCG CGC CCA CGC CTC GAG CCG ACC ATC CGC CTG CCG TCC GAG ′3 (SEQ ID NO:50) and 5′GGA TCC GAC GGC GTC GTC CGT TTC GTC GCC GGC CGC CGC CGC GGC GCG CTC GGA CGG CAG GCG GAT GG 3′ (SEQ ID NO:51) were used in an initial PCR reaction.

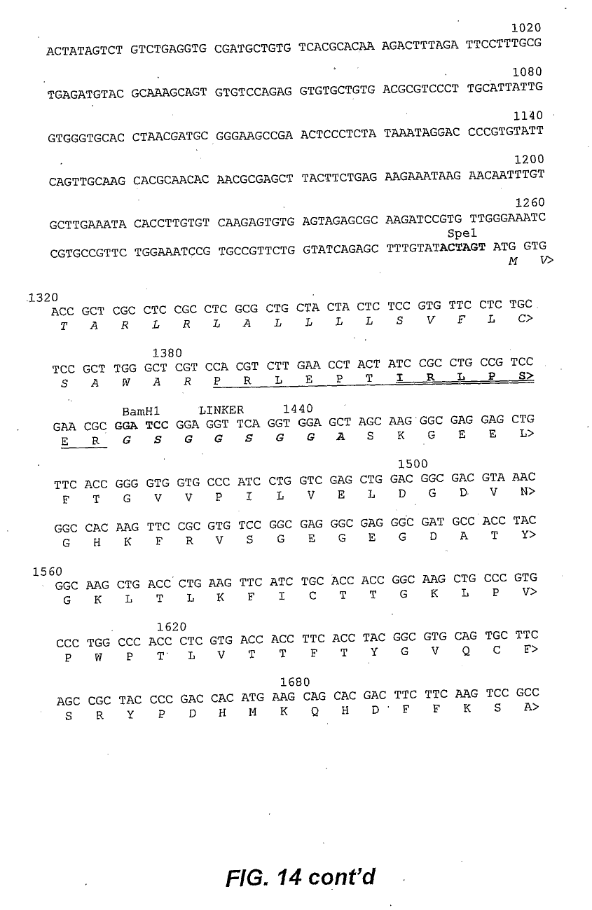

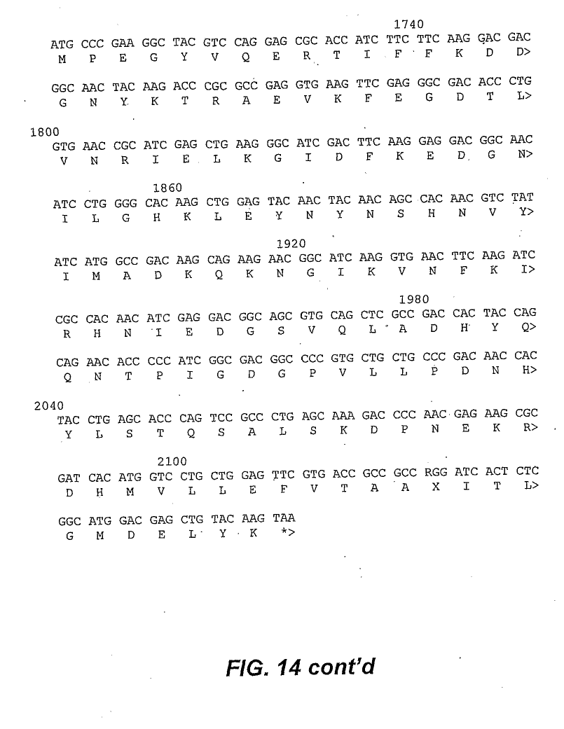

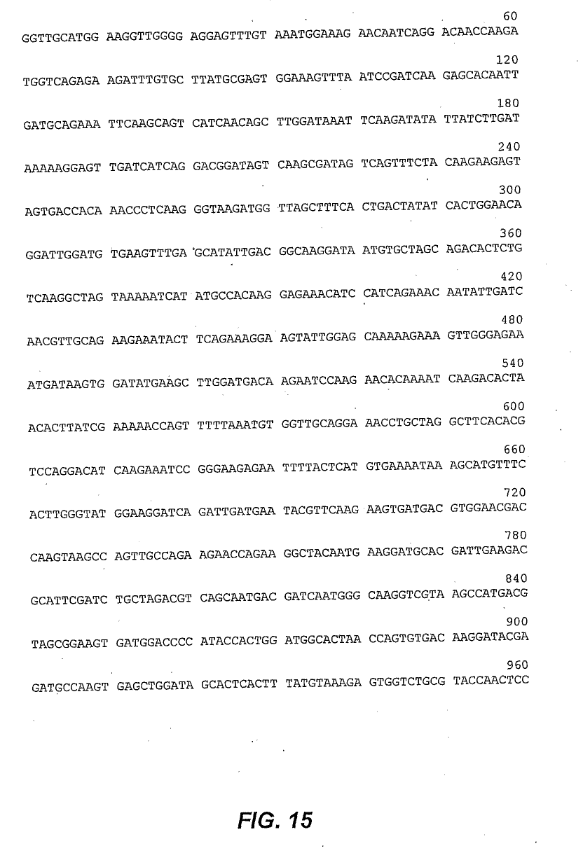

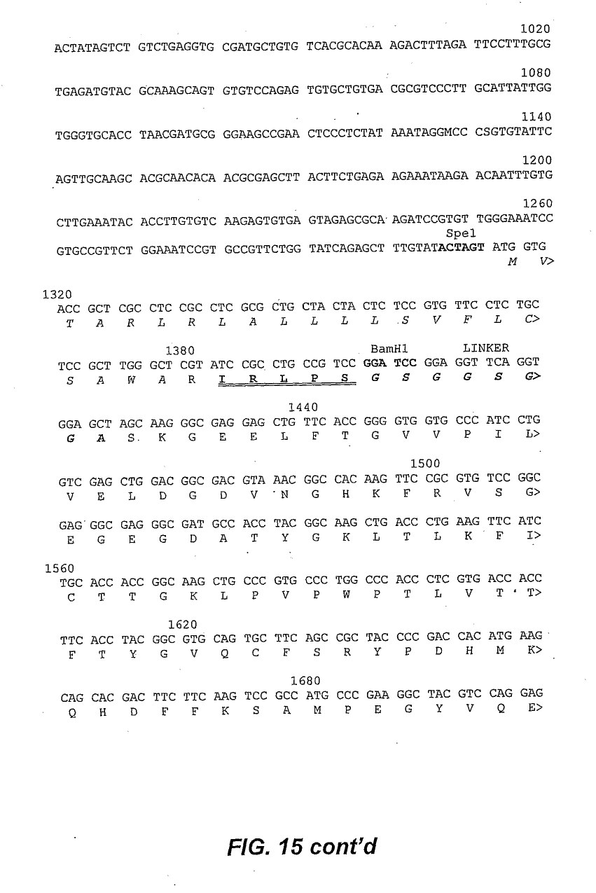

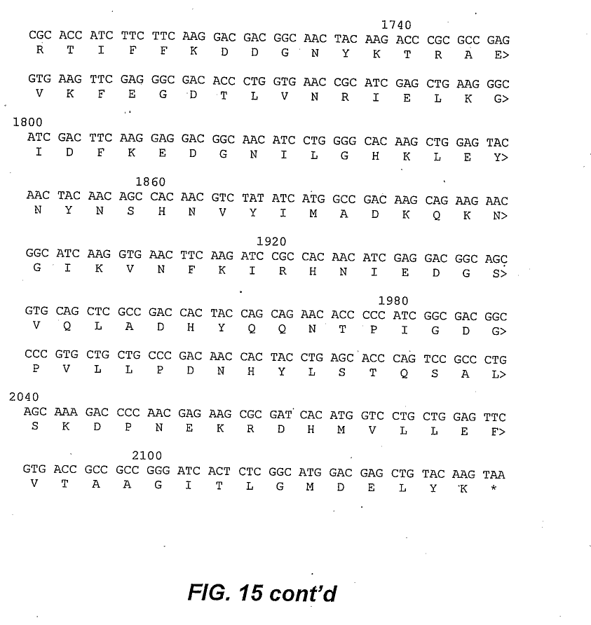

A subsequent PCR reaction using template from the 1st was performed using the primers SigF and Exp1Rev#3 consisting of the sequences 5′ACT AGT ATG GTG ACC GCT CGC CTC CGC CTC GCG CTG CTA CTA CTC TCC GTG TTC CTC TGC TCC GCG TGG GCG CGC 3′ and 5′GGA TCC GAC GGC GTC GTC CGT TTC GTC 3′ respectively. Incorporation of the restriction sites Spe1 and BamH1 (italics) allowed cloning into vector pCvpst5-gfp which was kindly provided by Dr Frank Smith, CSIRO Plant Industry, Queensland BioSciences Precint, 306 Carmody Rd., St Lucia Qld 4067. This vector was prepared with a BamH1 site adjacent a small amino acid linker (GGSGGAS) (SEQ ID NO:52) fused to the second amino acid of the S65T version of GFP. A similar cloning strategy was used for pCvEndoexp2-gfp (SEQ ID NO: 56 & FIG. 14) and pCvEndoexp3-gfp (SEQ ID NO: 57 & FIG. 15) except only one overlapping PCR reaction was required. In both cases primer SigF was used (as above). For pCvEndoexp2 the reverse primer Exp2 Rev#1 consisted of the sequence 5′GGA TCC GCG CTC GGA CGG. CAG GCG GAT GGT CGG CTC GAG GCG TGG GCG CGC CCA CGC GGA GCA GAG GAA 3′ (SEQ ID NO:58). For pCvEndoexp3 the reverse primer Exp3 Rev#1 consisted of the sequence 5′ GGA TCC GGA CGG CAG GCG GAT GCG CGC CCA CGC GGA GCA GAG GAA 3′ (SEQ ID NO:59). The BamH1 site incorporated for cloning of both primers Exp2 Rev#1 and Exp3 Rev#1 into pCvpst5-gfp is italicized in the sequence shown above.

### Growth and Transformation of Sugarcane Callus

Callus was initiated from Q117 meristematic tissue using the methods described by (Franks and Birch, 1991). Callus cells were maintained on MSC3 medium at 28° C. in the dark and subcultured every two weeks. Q17 suspension cells were initiated from callus cells and grown in liquid MSC3 medium with shaking at 60 rpm also in the dark at 28° C.

Callus was transformed by microprojectile bombardment with plasmid DNA following the method of (Bower et al., 1996). Tissue was co-bombarded with plasmid pEMU, which encodes the nptII gene conferring antibiotic resistance, under the control of the maize adh1 promoter (Last et al., 1991). Regeneration of plants was initiated by eliminating the synthetic auxin (IAA) from the growth medium and exposure of the callus to continuous light.

### Microscopy and Cytochemistry

GFP fluorescence monitoring in callus cultures, cells examination and the taking of photographs were performed as described in Example 1 above.

Confocal images were obtained using a Zeiss LSM 510 Meta confocal microscope.

In addition to the stains purchased and used as discussed in Example 1 above, the following stains were purchased from Molecular Probes (Invitrogen, Mt Waverley, Vic. 3149, Australia) and used according to the manufacturers instructions: the vacuolar lumen marker/protease substrates, CMAC-Arg (7-amino-4-chloromethylcoumarin, L-arginine amide) and CMAC-Ala-Pro (7-amino-4-chloromethylcoumarin, L-alanyl-L-proline amide); the pH sensitive Lysosensor Yellow/Blue DND160; DAPI nucleic acid stain; and propidium iodide.

### Transient Assays in Diverse Species

Fresh plant material was obtained from a local supermarket. Sections were prepared and placed on filter paper moistened with 50 mM sodium phosphate buffer ph 6.5. Plasmid DNA representing pCvEndoexp1-gfp and pCvgfpt were precipitated onto tungsten particles and tissues bombarded at 2000 psi using the helium pulsed gene gun. Tissues were kept moist and placed in the dark at room temperature for 48 hours at which time GFP expression was monitored using a Zeiss Axioskop epifluorescence microscope (Carl Zeiss Australia, North Ryde, NSW, 2133)

### Analysis of Sugarcane Transgenic Plants

Sugarcane transgenics representing both putative targeted GFP lines (pCvEndoNTPP-gfp) together with cytosolic GFP lines (pCvgfpt) were regenerated and grown in glasshouse conditions at 30° C. for 11 months. Fully mature plants were analysed for GFP localisation using a Zeiss LSM 510 Meta confocal microscope. Routinely, the tissue analysed included sections from internodes 2, 4 and 8, young leaf, old leaf and roots.

To enable gfp fluorescence to be observed in the highly acidic and proteolytic vacuolar compartments, sugarcane sections were treated 48 hours prior to microscopy with the following inhibitors:
Papain specific cysteine protease inhibitor (e64d) at 50 mM
A cocktail of protease inhibitors (Roche)
ConcanamycinA at 1 mM.

### EXAMPLE 2

### Identification of Candidate Gene

The endopeptidase encoded by EST MCSA201C03 is a member of the legumain family of cysteine proteases (clan CD, family C13) with a cleavage specificity for the carboxy side of asparagine residues (Chen et al. 1998). Legumains are also known as vacuolar processing enzymes (VPE) as, with the exception of a single cell wall representative from barley (Linnestad et al. 1998), they all occur in the vacuole (Müntz et al. 2002). γVPE from Arabidopsis has been localized to the lytic vacuole by electron microscope immuno-gold labeling (Kinoshita et al. 1999). VPEs are thought to be transported to the vacuole in vesicles in an inactive form and then auto-catalytically processed to an active form in the acidic environment of the vacuole. VPEs are also thought to have a role in the proteolytic activation of other classes of cysteine protease within the vacuole. In sugarcane, microarray experiments have shown that this sequence is strongly up-regulated as the stem matures (Casu et al. 2004).

### EXAMPLE 3

### Bioinformatic Analysis of Putative Domains in Sugarcane Sequence

The EST encoding the sugarcane endopeptidase (MCSA201C03) includes about 1 kb of sequence from the 3′ end of the gene. The investigators used this sequence together with other sugarcane sequences from public databases to construct a hypothetical complete endopeptidase sequence. This hypothetical sequence was used to predict primer sequences to generate a full-length clone from sugarcane stem cDNA by PCR. The products of the PCR were cloned into pGEMT and sequenced. The amino acid sequence encoded by this clone is shown in FIG. 1. Analysis with the Signal P program (V2.0) predicts that the sequence includes an N-terminal peptide, with predicted cleavage site between amino acid residues 22 and 23 (FIG. 1).

The N-terminal amino acid sequence of a homologue from Vigna, VmPE-1, has been determined experimentally. This suggests that residues 23 to 47 comprise an N-terminal propeptide which is removed during maturation of the protein (Linnestad et al. 1998; Okamoto and Minamikawa 1999). In the sugarcane protein, two aspartic acid residues precede the predicted cleavage site, suggesting that an aspartic endopeptidase could be involved in processing, FIG. 1.

By analogy with the barley aleurain (another cysteine protease), this N-terminal propeptide may comprise the vacuolar targeting element. Within the putative propeptide of the endopeptidase is a highly conserved domain consisting of the sequence -IRLPS- (SEQ ID NO: 2) in sugarcane, with conservative substitutions in other species (I/L)(R/K)(L)(P)(S) (SEQ ID NO: 24) (FIG. 3). The conserved topology appears to be “hydrophobic-charged-hydrophobic-proline-hydrophilic”, wherein “hydrophobic” preferably comprises an amino acid selected from the group consisting of: glycine, alanine, valine, leucine and isoleucine; “charged” preferably comprises an amino acid selected from the group consisting of: lysine, arginine and histidine; and “hydrophilic” preferably comprises an amino acid selected from the group consisting of: serine, threonine, asparagine and glutamine. This motif is found in the putative propeptide of plant legumain homologues, but not in animal homologues. A consensus sequence derived from sequences shown in FIG. 3 comprises amino acids: MVXXRLRLALLLXXXXLCSAWARPRLEPTIRLPSERAAA (SEQ ID NO: 37), wherein X may be any amino acid or deletion, but preferably is a corresponding amino acid as shown for Sc (SEQ ID NO: 10) or Zm (SEQ ID NO: 11) in FIG. 3. This consensus sequence, or fragment or selected amino acids thereof may comprise vacuole targeting elements, including for example, IRLPS (SEQ ID NO: 2).

Examination of the sequences of other sugarcane proteins revealed that this conserved motif is also found in three other proteins which are predicted to reside in the vacuole; a carboxypeptidase, a trypsin inhibitor protein and an aspartic protease, as shown in FIG. 4. Although these proteins have little other sequence homology, they all contain the conserved motif in a similar position at the N-terminal end of the protein (FIG. 4). Because of the conservation of the sequence and the strong link with vacuolar localization, this motif was considered to be a good candidate for testing as a vacuolar targeting element.

Within the sugarcane asparaginyl endopeptidase sequence, a putative C-terminal propeptide was also identified (see FIG. 1). Cleavage of this C-terminal peptide in the acidic environment of the vacuole probably activates the protease (Kuroyanagi et al. 2002).

### EXAMPLE 4

### Expression of Reporter Gene Constructs in Sugarcane Cells

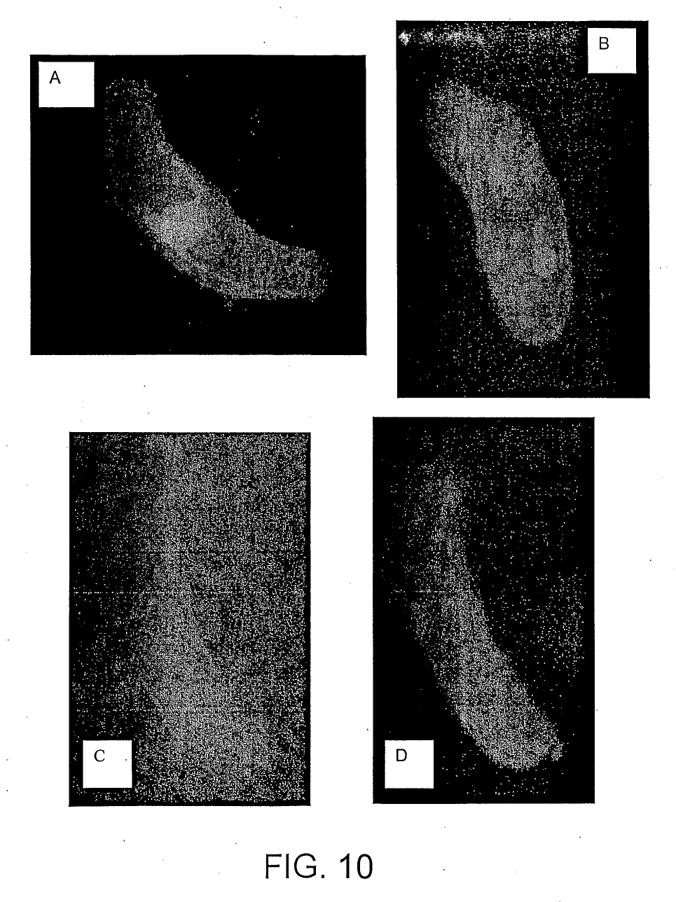

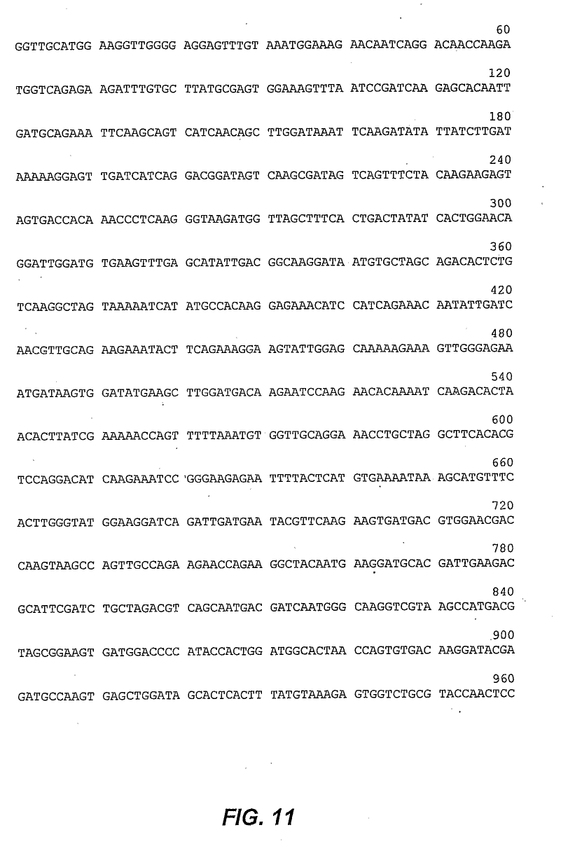

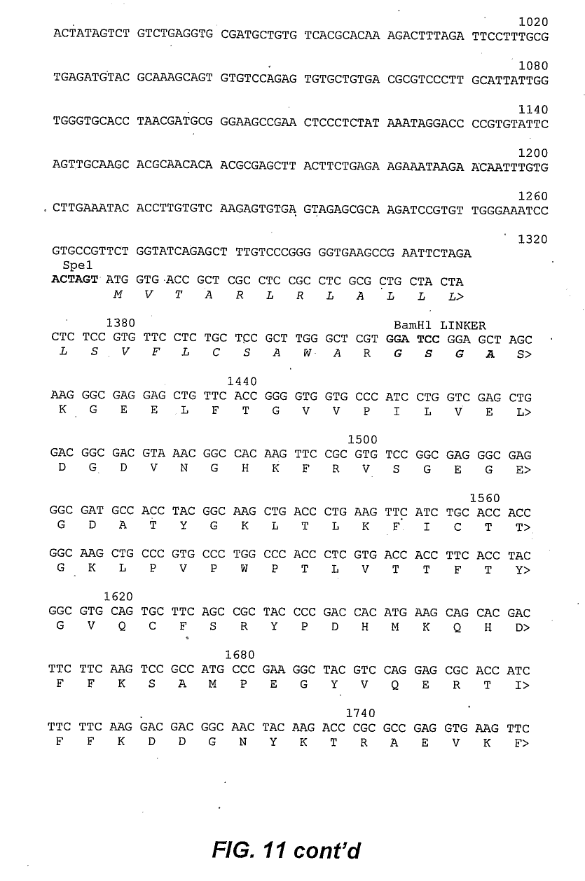

Sequence encoding the N-terminal region of the sugarcane asparaginyl endopeptidase gene was generated via PCR. The sequence consists of 264 nucleotides, encoding the secretory signal peptide, the putative vacuolar targeting motif and the first 40 amino acids of the mature protein. This sequence was fused to the green fluorescent protein (GFP) reporter gene in a vector under the control of the banana streak virus promoter (see FIG. 9). Sugarcane callus cells were transformed with this construct by particle bombardment as described herein. As a control, sugarcane callus cells were transformed with the same GFP vector without the addition of any putative targeting signal. Microscopic examination showed that the GFP was present in the cytoplasm and nucleus of the control cells (FIG. 10A). In contrast, in cells transformed with the construct comprising the targeting sequence, GFP was present in the central vacuole and absent from the nucleus and the peripheral cytoplasm (FIG. 10B).

The identification of this compartment as the vacuole was supported by labelling of sugarcane callus cells with a number of marker dyes with known localization patterns. The vacuole was identified by labelling with a fluorescent dye that is sequestered into the vacuolar lumen, CellTracker Blue CMAC (7-amino-4-chloromethyl-coumarin) (FIG. 10C) and with a dye that labels the tonoplast, MDY-64 (FIG. 10D). The pattern of fluorescence obtained with this dye was identical to that in the targeted GFP construct, suggesting that the GFP is accumulated in the vacuolar lumen.

### EXAMPLE 5

### GFP Reporter Analysis in Mature Sugarcane Transgenics

Transgenic sugarcane representing 7 pCvgfpt control lines and 17 pCvEndoNTPP lines comprising 264 nucleotides, encoding the secretory signal peptide, the putative vacuolar targeting motif and the first 40 amino acids of the mature protein were grown to maturity and analysed by confocal microscopy. PCR analysis of sugarcane genomic DNA using primers specific for GFP revealed that all plants contained the transgene.

Of the 7 pCvgfpt control lines, 5 lines showed good GFP expression with localisation to the cytoplasm and strong accumulation in the nucleus. Two lines that had shown GFP expression in callus appeared to be silenced in regenerated plants.

Of the 17 pCvEndoNTPP lines, 9 had some GFP fluorescence although intensity varied between lines, presumably due to the effects of variable insertion number and location. The remaining eight lines which had expressed GFP fluorescence in callus culture appeared to be silenced in regenerated plants.

In stem sections of both internode 4 and 8, GFP was localised to a large vacuolar compartment in the vascular parenchyma cells. Similar cells in root tissue also showed strong vacuolar fluorescence.

In the stem parenchyma cells, GFP was visible in a reticulate pattern throughout the whole cell in addition to some labelling of the nuclear envelope. This pattern is consistent with localisation in the endoplasmic reticulum. Small vesicle-like structures also showed GFP fluorescence. These appeared to be connected to the ER network and probably represent the Golgi apparatus or transfer vesicles. There was no co-localisation of the cell wall stain propidium iodide with GFP, indicating that no GFP was being secreted from the cells. This evidence suggests that the GFP fusion protein is being processed correctly through the ER and Golgi apparatus but that the GFP inside the large vacuolar compartment is short-lived due to the intense proteolytic and acidic nature of these compartments.

To test this hypothesis, a series of protease inhibitors together with a proton pump inhibitor (concanamycinA) were used. In recent studies of vacuole targeting in Arabidopsis, the addition of the cysteine protease inhibitor e64d caused a dramatic change to gfp stability in the vacuole. In the current study, the addition of e64d and a broad range of protease inhibitors resulted in no difference in gfp stability in the sugarcane vacuole. The addition of a proton pump inhibitor (ConcanamycinA) however, caused a dramatic effect. After 48 hours immersion, gfp could now be observed in the large vacuoles throughout the storage parenchyma cells. Fluorescence was however at a lower intensity then that seen in the vacuolar parenchyma cells. In leaf tissue submerged in concanamycinA, gfp was observed in large vacuoles throughout the epidermal cells as well as in guard cells. The mesophyll cells showed strong red autofluorescence from the chlorophyll and no observable gfp fluorescence. There was no mistargeting of gfp to the chlorophyll as was seen in recent sugarcane vacuolar studies using the NPIR like targeting signal from sweet potato sporaminin (Gnanasambandam and Birch 2004). These results suggest that the targeting element identified within the endopeptidase gene is functional within most cell types and that the gfp reporter system is highly sensitive to pH fluctuations.

### EXAMPLE 6

### Testing of the NTPP Region for Vacuole Targeting Ability in Sugarcane

GFP fusion constructs were prepared to pinpoint the vacuole-targeting motif identified in the NTPP of the sugarcane endopeptidase gene. Co-bombardment of sugarcane callus tissue with plasmid pEMU allowed for the selection of stable transgenic callus lines.

Constructs pCvsgfp and pCvsgfpKDEL were designed to label the apoplastic space and the endoplasmic reticulum respectively. Both constructs contained the endopeptidase signal peptide which functions to promote translation of GPF into the endomembrane system. In addition to the signal peptide, an ER retention motif (KDEL) was incorporated at the C terminus of GFP in construct pCvsgfpKDEL.

GFP fluorescence was localised mainly to the apoplastic space in pCvsgfp lines A faint labelling of the ER system can also be observed in some cells. In contrast bright labelling of the ER system and no apoplastic labelling was evident from lines carrying pCvsgfpKDEL. Optically sectioning through the callus by confocal microscopy revealed a reticulate GFP pattern characteristic of the ER membrane structure. Callus lines containing plasmid pcvgfpt alone with no additional targeting information showed fluorescence throughout the cytoplasm with concentration of signal in the cells nucleus. Cytoplasmic streaming of GFP was also sometimes evident.

Callus lines harbouring pCvEndoexp1, 2 and 3 and the original pCvEndoNTPP-gfp constructs all showed a predominant vacuolar GFP localisation pattern. No GFP fluorescence could be observed in the cytoplasm and nucleus indicating that GFP is being processed through the endomembrane system. The absence of GFP in the nucleus was confirmed by co-localisation studies using the nuclear stain DAPI. Furthermore, in all vacuole-targeted lines, no GFP could be observed in the intracellular spaces, indicating the presence of a positive sorting signal. No observable difference could be seen between pCvEndoExp1, 2 and 3, indicating that the addition of just the minimal targeting sequence IRLPS is sufficient for vacuolar targeting. In all constructs where GFP is delivered through the endomembrane system, some aberrant expression due to overloading was evident.

To confirm localisation of GFP to the vacuole, a series of fluorescent protease substrates were used to label the sugarcane vacuole. Co-localisation of vacuole-targeted GFP was achieved with the fluorescence protease substrates CMAC-Arg; CMAC-Ala-Pro and CellTracker Blue CMAC. Furthermore both the neutral red acidic marker and Lysosensor DND160 stained a similarly sized vacuolar compartment in Q117 sugarcane suspension cells, adding to the evidence that GFP is being correctly targeted to a large lytic and proteolytic vacuolar compartment in sugarcane.

### EXAMPLE 7

### Transient Expression of Vacuole-targeted GFP in Diverse Species

The endopeptidase gene is highly conserved among plant genera (FIG. 3), suggesting that this motif might be effective for vacuolar targeting in a wide range of species. The endopeptidase NTPP containing the vacuolar-targeting motif was tested for its targeting ability in diverse species using transient expression analysis. Constructs pCvEndoexp1-gfp and pcvgfpt were analysed in a range of tissues outlined in Table 2. The results showed that the vacuolar targeting element from sugarcane was effective in a wide range of phylogenetically diverse species including both dicots, and monocots.

throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

The disclosure of each patent and scientific document, computer program and algorithm referred to in this specification is incorporated by reference in its entirety.

### REFERENCES

Altschul, S. F., Gish, W., Miller, W., Meyers, E. W. and Lipman, D. J. (1990) Basic local alignment search tool. J. Mol. Biol. 215, 403-410.
Bassham, D. C. and Raikhel, N. V. (2000) Unique features of the plant vacuolar sorting machinery. Curr. Op. Cell Biol. 12, 491-495.
Bower, R., Elliott, A. R., Potier, B. A. M. and Birch, R. G. (1996) High-efficiency, microprojectile-mediated cotransformation of sugarcane, using visible or selectable markers. Molec. Breeding 2: 239-249.
Casu, R. E., Grof, C. P. L., Rae, A. L., McIntyre, C. L., Dimmock, C. M. and Manners, J. M. (2003) Identification of a novel sugar transporter homologue strongly expressed in maturing stem vascular tissues of sugarcane by expressed sequence tag and microarray analysis. Plant Molec. Biol. 52, 371-386.
Casu, R. E., Dimmock, C. M., Chapman, S. C., Grof, C. P. L., McIntyre, C. L., Bonnett, G. D. and Manners, J. M. (2004) Identification of differentially expressed transcripts from maturing stem of sugarcane by in silico analysis of stem expressed sequence tags and gene expression profiling. Plant Molec. Biol. 54, 503-517.
Chamberlain, D. A., Brettell, R. I. S., Last, D. I., Witrzens, B., McElroy, D., Dolferus, R. and Dennis, E. S. (1994) The use of the Emu promoter with antibiotic and herbicide resistance genes for the selection of transgenic wheat callus and rice plants Aust. J. Plant Physiol. 21: 95-112.
Chen, J.-M., Rawlings, N. D., Stevens, R. A. E. and Barrett, A. J. (1998) Identification of the active site of legumain links it to caspases, clostripain and gingipains in a new clan of cysteine endopeptidases. FEBS Lett. 441, 361-365.
Gnanasambandam, A. and Birch, R. G. (2004) Efficient developmental mis-targeting by the sporamin NTPP vacuolar signal to plastids in young leaves of sugarcane and Arabidopsis. Plant Cell Reports 23, 435-447.
Jacobsen, K. R., Fisher, D. G., Maretzki, A. and Moore, P. H. (1992) Developmental changes in the anatomy of the sugarcane stem in relation to phloem unloading and sucrose storage. Botanica Acta 105, 70-80
Kinoshita, T., Yamada, K., Hiraiwa, N., Kondo, M., Nishimura, M. and Hara-Nishimura, I. (1999) Vacuolar processing enzyme is up-regulated in the lytic vacuoles of vegetative tissues during senescence and under various stressed conditions. Plant J 19, 43-53.
Kuroyanagi, M., Nishimura, M. and Hara-Nishimura, I. (2002) Activation of Arabidopsis vacuolar processing enzyme by self-catalytic removal of an auto-inhibitory domain of the c-terminal propeptide. Plant Cell Physiol. 43, 143-151.
Linnestad, C., Doan, D. N. P., Brown, R. C., Lemmon, B. E., Meyer, D. J., Jung, R. and Olsen, O.-A. (1998) Nucellain, a barley homolog of the dicot vacuolar-processing protease, is localized in nucellar cell walls. Plant Physiol. 118, 1169-1180.
Müntz, K., Blattner, F. R. and Shutov, A. D. (2002) Legumains—a family of asparagines-specific cysteine endopeptidases involved in proprotein processing and protein breakdown in plants. J. Plant Physiol. 159, 1281-1293.
Neuhaus, J.-M. and Rogers, J. C. (1998) Sorting of proteins to vacuoles in plant cells. Plant Molec. Biol. 38, 127-144.
Okamoto, T. and Minamikawa, T. (1999) Molecular cloning and characterization of Vigna mungo processing enzyme 1 (VmPE-1), an asparaginyl endopeptidase possibly involved in post-translational processing of a vacuolar cysteine endopeptidase. Plant Molec. Biol. 39, 63-73.
Schenk et al. (2001) Promoters for pregenomic RNA of banana streak badnavirus are active for transgene expression in monocot and dicot plants. Plant Molec. Biol. 47, 399-412
Vitale, A. and Raikhel, N. V. (1999) What do proteins need to reach different vacuoles? Trends Plant Sci. 4, 149-155.

TABLE 1
Description of the asparaginyl endopeptidases shown in
FIG. 3 and respective corresponding protein accession numbers
in Genpept (GP) or SwissProt (SP) and nucleic acid accession
numbers in GenBank.

| | | | Nucleic acid |
|---|---|---|---|
| | | | database |
| | | Protein database | accession |
| | Description | accession number | number |
| Sc | sugarcane asparaginyl | | |
| | endopeptidase | | |
| Zm | Zea mays C13 | AAD04883 (GP) | AF082347 |
| | endopeptidase NP1 | | |
| | precursor | | |
| Os | Oryza sativa asparaginyl | NP_918390 (GP) | NM_193501 |
| | endopeptidase | | |
| At | Arabidopsis thaliana | VPEG_ARATH (SP) | D61395* |
| | vacuolar processing | | |
| | enzyme, gamma-isozyme | | |
| | precursor | | |
| Nt | Nicotiana tabacum vacuolar | BAC54828 (GP) | AB075948 |
| | processing enzyme-1b | | |
| Cs | Citrus sinensis vacuolar | VPE_CITSI (SP) | Z47793* |
| | processing enzyme | | |
| | precursor | | |
| Xl | Xenopus laevis MGC64351 | AAH56842 (GP) | BC056842 |
| | protein | | |
| Rn | Rattus norvegicus legumain | NP_071562 (GP) | NM_022226 |
| Bt | Bos taurus legumain | NP_776526 (GP) | NM_174101 |
| Hs | Homo sapiens legumain | LGMN_HUMAN | Y09862* |
| | precursor | (SP) | |

*notes nucleotide accession number is cross-referenced (xref) to the protein accession number.

TABLE 2
Description of the location of GPF expression with and without
endopeptidease vacuaolar targeting signal in diverse tissue types.

| | | | Location of | Location of |
|---|---|---|---|---|
| | | | GFP | GFP following |
| | | | following | expression of |
| | | Common | expression | pCvEndoExp1- |
| Species | Family | name | of pCvgfpt | gfp |
| Apium | Apiaceae | Celery | Cytoplasm | Lytic vacuole |
| graveolens | | | and nucleus | |
| Asparagus | Liliaceae | Asparagus | Cytoplasm | Lytic vacuole |
| officinalis | | | and nucleus | ER |
| Cucurbita | Cucurbitaceae | Zucchini | Cytoplasm | Lytic vacuole |
| pepo | | | and nucleus | |
| Gossypium | Malvaceae | Cotton | Cytoplasm | Lytic vacuole |
| hirsutum | | | and nucleus | |
| Zea mays | Poaceae | Maize | Cytoplasm | Lytic and |
| | | | and nucleus | storage |
| | | | | vacuoles |

1. A chimeric protein comprising:
(i) a vacuole targeting sequence X₁X₂X₃PX₄ (SEQ ID NO:1) wherein:
X₁ is a hydrophobic amino acid;
X₂ is a basic amino acid;
X₃ is a hydrophobic amino acid
P is proline; and
X₄ is a hydrophilic amino acid; and
(ii) an amino acid sequence of a heterologous protein which does not normally comprise said vacuole targeting sequence or which normally comprises a different vacuole targeting sequence;
arranged so that said vacuole targeting sequence is capable of facilitating targeting of the chimeric protein to a vacuole in a plant cell.

2. The chimeric protein of claim 1 wherein X₁ is isoleucine.

3. The chimeric protein of claim 1 wherein X₁ and/or X₃ is/are leucine.

4. The chimeric protein of claim 1 wherein X₂ is lysine or arginine.

5. The chimeric protein of claim 1 wherein X₄ is serine.

6. The chimeric protein of claim 1 wherein the vacuole targeting sequence is (I/L)(R/K)LPS (SEQ ID NO:24).

7. The chimeric protein of claim 1 wherein the vacuole targeting sequence is selected from the group consisting of: IKLPS (SEQ ID NO:3); LRLPS (SEQ ID NO:4); and LKLPS (SEQ ID NO:5).

8. The chimeric protein of claim 1, further comprising a secretory signal peptide.

9. The chimeric protein of claim 8, wherein the secretory signal peptide comprises an amino acid sequence selected from the group consisting of: MVTARLRLALLLLSVFLCSAWA (SEQ ID NO: 8); MRPAGQLLLPLLLLAVAASM (SEQ ID NO: 37); MRPAGQLLLPLLLLAVSVAAA (SEQ ID NO: 38); and MGTIPWIPAMLWALLWGATA (SEQ ID NO: 39).

10. The chimeric protein of claim 1 wherein the heterologous protein normally lacks a vacuolar targeting sequence.

11. The chimeric protein of claim 1, wherein the heterologous protein is selected from the group consisting of: a sucrose modifying enzyme, a hexose modifying enzyme, a protein capable of use as an industrial enzyme, a protein capable of use in a pharmaceutical composition, a protein capable of use as a diagnostic reagent, a protein capable of use in crop protection, a protein characterized by a culinary property, a protein characterized by an industrial property and a vacuolar metabolite modifying enzyme.

12. The chimeric protein of claim 11 wherein the sucrose modifying enzyme is selected from the group consisting of a sucrose isomerase, a fructosyl transferase, an invertase, an amylosucrase, a dextransucrase and a glucan sucrase.

13. The chimeric protein of claim 12 wherein the hexose modifying enzyme is capable of directly modifying a hexose structure.

14. The chimeric protein of claim 13 wherein the hexose modifying enzyme is selected from the group consisting of a polyol dehydrogenase, a dextran synthase and a other transferase protein.

15. The chimeric protein of claim 11 wherein the protein capable of use as an industrial enzyme is selected from the group consisting of a lipase, a cellulase, a pectinase, a hemicellulase, a peroxidase, an amylase, a dextranase, a protease, a polysaccharase, a lytic enzyme and other proteins.

16. The chimeric protein of claim 11 wherein the protein capable of use in a pharmaceutical composition is selected from the group consisting of an antigen, an antibody, an antibody fragment, a cytotoxic agent, an anticancer protein, an immunotherapeutic agent, a vaccine, an hormone, a cytokine and the like.

17. The chimeric protein of claim 11 wherein the protein capable of use as a diagnostic reagent is selected from the group consisting of an antigen, an antibody, an antibody fragment, a cytotoxic agent, an anticancer protein, an immunotherapeutic agent, a vaccine, an hormone, a cytokine and the like.

18. The chimeric protein of claim 11 wherein the protein capable of use in crop protection is selected from the group consisting of an antifungal protein, an antibacterial protein, an anti-insect protein and an anti-nematode protein.

19. The chimeric protein of claim 18 wherein the antifungal protein is a plant defensin.

20. The chimeric protein of claim 18 wherein the antibacterial protein comprises a thionin.

21. The chimeric protein of claim 18 wherein the anti-insect protein is selected from the group consisting of a Bos taurus legumain, a protease inhibitor and an avidin.

22. The chimeric protein of claim 18 wherein the anti-nematode protein comprises a collagenase.

23. The chimeric protein of claim 11 wherein the protein characterized by a culinary property comprises a property selected from the group consisting of a coagulant property, a gelling property, a sweet property, a sour property and an adhesive property.

24. The isolated protein of claim 11 wherein the protein characterized by an industrial property comprises a property selected from the group consisting of a coagulant property, a gelling property, a sweet property, a sour property and an adhesive property.

25. The chimeric protein of claim 11 wherein the vacuolar metabolite modifying enzyme comprises an enzyme capable of modifying a compound selected from the group consisting of a phenolic compound, a tannin compound, a flavonoid compound and another secondary metabolite.

26. The isolated protein of claim 25 wherein the vacuolar metabolite modifying enzyme modifies a vacuolar metabolite of a vacuole.

27. The isolated protein of claim 25 or claim 26 wherein the vacuolar metabolite modifying enzyme modifies a vacuolar metabolite of a monocotyledon plant.

28. The isolated protein of claim 25 or claim 26 wherein the vacuolar metabolite modifying enzyme modifies a vacuolar metabolite of a dicotyledon plant.

29. The isolated protein of claim 27 wherein the monocotyledon plant is selected from the group consisting of sugarcane plant, a maize plant, a wheat plant, a barley plant, a sorghum plant, a rye plant, an oat plant and a rice plant.

30. An isolated nucleic acid encoding the chimeric protein of claim 1.

31. The isolated nucleic acid of claim 30, which encodes a vacuole targeting sequence is selected from the group consisting of: IKLPS (SEQ ID NO:3); LRLPS (SEQ ID NO:4); and LKLPS (SEQ ID NO:5).

32. The isolated nucleic acid of claim 30 or claim 31, which further encodes a secretory signal peptide.

33. A genetic construct that comprises an isolated nucleic acid encoding the vacuolar targeting sequence set forth in SEQ ID NO:1.

34. The genetic construct of claim 33, wherein the isolated nucleic acid further encodes a heterologous protein.

35. The genetic construct of claim 33 or claim 34, which is an expression construct comprising an expression vector, wherein said isolated nucleic acid is operably linked to one or more regulatory elements present in said expression vector.

36. A method of producing a genetically modified plant including the step of introducing the isolated nucleic of claim 30 into a plant cell or tissue.

37. The method of claim 36, further including the step of selectively propagating a genetically-modified plant from said plant cell or tissue.

38. The method of claim 36 or claim 37, wherein the isolated nucleic acid is present in an expression construct.

39. The method of claim 38, wherein the plant cell or tissue is callus.

40. The method of claim 39, wherein the plant is a dicotyledon.

41. The method of claim 39, wherein the plant is a monocotyledon.

42. The method of claim 41, wherein the monocotyledon is selected from the group consisting of sugarcane plant, a maize plant, a wheat plant, a barley plant, a sorghum plant, a rye plant, an oat plant and a rice plant.

43. A genetically modified plant comprising the isolated nucleic acid of claim 32.

44. The genetically modified plant of claim 42, which is a dicotyledon.

45. The genetically modified plant of claim 42, which is a monocotyledon.

46. The genetically modified plant of claim 45, wherein the monocotyledon is selected from the group consisting of sugarcane plant, a maize plant, a wheat plant, a barley plant, a sorghum plant, a rye plant, an oat plant and a rice plant.

47. A tissue, cell, organelle or other part obtainable from the genetically modified plant of claim 46.

48. The organelle of claim 47, which comprises a vacuole.

49. The organelle of claim 48, wherein the vacuole is a lytic vacuole.

50. The tissue, cell, organelle or other part of claim 47, which is a plant part selected from a fruit, a leaf, a root, a shoot, a stem, a flower, a seed, a cutting or other reproductive material.

51. A method for producing a recombinant protein in a plant including the steps of:
(1) expressing the chimeric protein of claim 1 in a plant; and
(2) isolating the expressed chimeric protein from a tissue, cell or organelle of said plant.

52. The method of claim 51 wherein the chimeric protein is isolated from an organelle of said plant.

53. The method of claim 52 wherein the organelle is a vacuole.

54. The method of claim 53 wherein the vacuole is a lytic vacuole.

55. A method for tissue specific expression of a chimeric protein in a plant including the step of expressing the isolated nucleic acid of claim 32 in a plant, whereby a chimeric protein encoded by the isolated nucleic acid is selectively targeted to a vacuole of said plant.

56. The method of claim 55, wherein the vacuole is a lytic vacuole.

57. The method of claim 55, wherein the plant is a dicotyledon.

58. The method of claim 57,

59. The method of claim 55, wherein the plant is a monocotyledon.

60. The method of claim 59, wherein the monocotyledon is selected from the group consisting of sugarcane plant, a maize plant, a wheat plant, a barley plant, a sorghum plant, a rye plant, an oat plant and a rice plant.
